(19) United States
(12) Patent Application Publication
(10) Pub. No.: US 2004/0197913 A1
(43) Pub. Date: Oct. 7, 2004

(54) Methods and means for improving retroviral integration

(76) Inventors: Mark James O'Connor, Cambridge (GB); Stephen Philip Jackson, Cambridge (GB); Alan Lau, Cambridge (GB)

Correspondence Address: BOZICEVIC, FIELD & FRANCIS LLP, 200 MIDDLEFIELD RD, SUITE 200, MENLO PARK, CA, 94025, US

(21) Appl. No.: 10/658,315

(22) Filed: Sep. 8, 2003

Related U.S. Application Data
(60) Provisional application No. 60/409,309, filed on Sep. 6, 2002.

(51) Int. Cl. A61K 48/00; C12N 15/867

(52) U.S. Cl. 435/456; 424/93.21

(57) ABSTRACT

Retroviral integration is promoted in mammalian cells by inhibition of RAD52 DNA-binding activity, e.g. using RNAi. This is useful in gene therapy, especially ex vivo. Retroviral integration is inhibited by increasing mammalian RAD52 DNA-binding activity, useful in inhibiting retroviruses.

The present invention relates to promoting retroviral integration in mammalian cells. It further relates to promoting retroviral integration in gene therapy, especially ex vivo.

The invention has arisen from the inventors' surprising finding that inhibiting RAD52 in mammalian cells allows for a substantial (e.g. 10 to 16-fold) increase in retroviral integration, despite the absence of any previously recognised severe phenotype being observed by knock-out of RAD52 in mammalian cells. The newly observed effect was not predictable from the available art, as is now explained with reference to background art.

Reverse transcription of retroviral RNA into linear double-stranded DNA and its subsequent integration into the host cell genome are essential steps in the retroviral life cycle. The non-homologous end-joining (NHEJ) DNA repair pathway has been implicated in protecting cells from retrovirus-induced apoptosis caused by either unintegrated linear viral double-stranded DNA or through host cell DNA damage produced during retroviral infection. It has previously been established that inhibition of this pathway is useful in inhibiting retroviral and retrotransposon activity. See e.g. Downs and Jackson, 1999, WO98/30903.

Eukaryotes have both NHEJ and homologous recombination (HR) repair pathways that can repair DNA double-strand breaks (DSB). However, significant differences have been found between the importance of HR in yeast and mammalian cells.

In yeast cells, repair of DNA DSBs occurs predominantly through the HR pathway. Components of the yeast HR pathway collectively constitute the RAD52 epistasis group. The RAD52 epistasis group consists of 10 genes and encodes proteins that include the yeast proteins Rad51p and RAD52p (Paques and Haber, 1999). Knockout-out or loss of function of any of the RAD52 epistasis group members results in viable yeast and all show defects in HR and hypersensitivity to DNA damaging agents such as ionizing radiation (1R). Mutants of Rad51p, RAD52p and Rad54p show the most severe recombination and repair defects, indicating that these proteins play crucial roles in HR (Petes et al., 1991; Game et al., 1993). In particular, RAD52p is the single most important component of yeast HR pathways and is absolutely required for all HR events (Paques and Haber, 1999).

Although functional homologs of yeast Rad51p, RAD52p and Rad54p exist in mammalian cells, clear differences in HR pathways between the two organisms have become apparent. In complete contrast to yeast, knockout of RAD52 in mammalian cells leads to only mild reductions in HR frequency (30-40%) and no obvious DNA repair defects. Moreover, RAD52 null adult mice were viable and showed no gross abnormalities whatsoever when compared to RAD52 positive mice (Rijkers et al., 1998). Differences in HR pathways between yeast and mammalian systems can be further highlighted by the fact that loss of Rad51 is lethal in mammalian cells. Although yeast RAD51 mutants are viable, targeted deletion of RAD51 in mice leads to early embryonic lethality (Tsuzuki et al., 1996; Lim et al., 1996). Similarly, conditional RAD51 mutants in chicken DT40 cells show extensive genetic instability and rapid cell death. The high incidence of chromosomal breaks that occurred during mitosis in RAD51 mutant chicken cells suggests that, unlike yeast, Rad51 plays a critical role in mammalian HR and replication fork progression (Sonoda et al., 1998).

Van Dyck et al. found that RAD52, like Ku, binds double-strand breaks in DNA caused by ionizing radiation and proposed a model in which either Ku or RAD52 binds, with Ku directing double-strand breaks into the NHEJ repair pathway and RAD52 initiating repair by homologous recombination. As a result of their results, Van Dyck et al. suggested that Ku and RAD52 direct entry into alternative pathways for repair, and further proposed that simultaneously overexpressing RAD52 while down-regulating or inactivating Ku would be useful in promoting the frequency of homologous gene targeting using linear DNA vectors.

Li et al. (2001) reported that unintegrated retroviral cDNA is a substrate for NHEJ pathway, showing that Ku70 and Ku80 bind to retroviral replication intermediates. This is consistent with the prior knowledge that inhibition of the NHEJ repair pathway, e.g. by targeting of the component Ku, has been shown to have efficacy in inhibiting TY1 retrotransposition in yeast and retroviral activity in mammalian cells (Downs and Jackson, 1999, WO98/30903). The comparable results for inhibiting such activity as between yeast and mammalian cells are consistent with the fact that knocking out NHEJ has a severe and comparable phenotype on both yeast and mammalian cells.

In contrast to NHEJ, Rattray et al. (2000) found that loss of any of the components of the HR pathway (RAD52 epistasis group) did not result in inhibition of yeast TY1 retrotransposition. In fact increases in retrotransposition rates were observed in HR deficient yeast cells, with mutants of RAD51 and RAD52 showing 11-fold and 24-fold increases respectively.

Until now, no experimental evidence for any role of the HR pathway or any of its components has been described for retroviral integration in mammalian systems. Moreover, given the absence of any severe phenotype resulting from RAD52 knock-out in mammalian cells (Rijkers et al., 1998), in contrast to the severe phenotype in yeast cells (Game et al., 1993), no role for the HR pathway or any one component of this pathway in particular in retroviral integration could have been envisaged.

The present invention arises from work in which the present inventors have created a number of mutations to knockout individually different components within the HR pathway in mammalian cells. The inventors tested the various mutant and knockout cells for susceptibility to integration of retroviral DNA into the genome.

RAD51 is thought to be a critical component of the mammalian HR pathway and knockout of RAD51 results in cellular lethality. The lack of viable RAD51 knockout cells prevents detailed analysis, however mammalian cells also contain several paralogs and regulators of Rad51 function. Paralogs of Rad51 include the XRCC2 and XRCC3 proteins (Thomson and Schild, 2001) and the breast cancer associated protein, BRCA2, which is implicated in mediating HR (Davies et al., 2001; Moynahan et al., 2001). Mammalian cells with loss of XRCC2, XRCC3 and BRCA2 are viable but show significantly reduced HR efficiency, chromosomal instability and sensitivity to DNA damaging agents (Takata et al., 2001; Thomson and Schild, 2001).

As described herein, the inventors' experimental analysis of mammalian cells lacking functional XRCC2, XRCC3 or BRCA2 showed no effects on retroviral transduction efficiencies. This result indicates that the HR pathway, in stark contrast to what is observed for yeast TY1 retrotransposition, is not directly involved in modulating the efficiency of retroviral integration in mammalian cells.

No effect on retroviral transduction efficiency was found in cells with loss of key components of the mammalian HR pathway, including XRCC2, XRCC3 and BRCA2. However, completely surprisingly, the inventors found that knocking out RAD52 in mammalian cells, e.g. by targeted deletion or RNAi, resulted in a substantial increase in retroviral integration within those cells. Not only was the fact of an increased susceptibility itself surprising, given that no other severe phenotype has been associated with the loss of mammalian RAD52 (Rijkers et al., 1998); the level of increase was remarkable—up to 16 fold over wild type cells. This observation is the most dramatic phenotype shown for RAD52 deficient cells seen to date.

As mentioned, the inventors have demonstrated that the enhancement of retroviral integration in mammalian cells does not arise from defects in the general HR repair pathway. Loss of components in the HR pathway, apart from RAD52, did not show any effect. Furthermore, the inventors have demonstrated experimentally that the role of RAD52 in inhibiting retroviral integration is independent of the role it may play in HR and is likely mediated via its ability to bind DNA. Overexpression experiments described herein show that RAD52 alone is sufficient to repress retroviral transduction efficiency and this effect is consistent with the role RAD52 plays in modulating retroviral integration. The use of DNA-binding or Rad51/RPA-interaction domain mutants of RAD52 show that DNA-binding activity, but not Rad51/RPA interactions, is required for the inhibition of retroviral integration. These overexpression experiments confirm that HR plays no active part in modulating retroviral integration and also indicate the DNA binding activity of RAD52 elicits these effects.

The huge increase in retroviral integration that is achieved by inhibiting RAD52 activity in mammalian cells makes it feasible at last to use retroviral targeting of therapeutic genes, especially in ex vivo therapies making use of explanted stem cells or other cells for retransplantation. (Thus, for example, stem cells may be removed from a person, treated and returned to that person, e.g. an adult giving informed consent or a child for which appropriate consent has been given.) Although numerous approaches are being tried in the field, levels of retroviral integration that have been achieved previously have been prohibitively low (e.g. see Hawley 2001). The fact that the present invention allows for increases in retroviral integration by a factor up to 16 or more opens up very exciting possibilities for retroviral therapy. Various aspects and embodiments of the present invention capitalise on this, for instance by inhibiting the ability of RAD52 protein to assemble or to bind to DNA, or by inhibiting expression of RAD52 protein in the cells. The use of RNAi represents a preferred embodiment for inhibiting RAD52 production in mammalian cells in a reversible manner.

In addition, the inventors' demonstration that overexpressing RAD52 or a DNA-binding fragment thereof in mammalian cells inhibited retroviral integration has potential in anti-retroviral therapy. Various further aspects and embodiments of the present invention are directed to this.

### BRIEF DESCRIPTION OF THE FIGURES

FIG. 1 shows results demonstrating the sensitivity and broad utility of HIV-1 based luciferase integration assays (LUCIA).

FIG. 1A is a highly schematic representation of HIV-1 LUCIA. After virus attachment and entry, viral RNA is reverse transcribed into a dsDNA copy by HIV-1 reverse transcriptase (RT). Viral dsDNA is then joined to host cell chromosomal DNA by HIV-1 integrase (IN). The viral integration process results in chromosomal DNA strand breaks and cellular DNA repair pathways repair this damage. Successful integration and repair results in expression of the luciferase reporter gene.

FIG. 1B shows results of HIV-1 LUCIA in Hela cells following infection with increasing quantities of packaged (VSVG⁺) or non-packaged (VSVG⁻) HIV vector.

FIG. 1C shows results of HIV-1 LUCIA in Hela cells following infection with increasing quantities of integrase proficient (IN⁺) or integrase defective (D64V IN⁻) HIV vector.

Data are given as the average luminescence (cps) from at least four wells of an opaque-white 96-well plate. In all cases the amount of IN⁺/VSVG⁺ virus added is directly proportional to the luciferase signal generated.

FIG. 2 shows results of experiments demonstrating that cells deficient in RAD52 show enhanced retroviral infection.

FIG. 2A shows HIV-1 LUCIA results for XRCC2 (IRS1) and XRCC3 (IRS1-SF) defective hamster cell lines infected with HIV-1 IN+ retrovirus stocks.

FIG. 2B shows HIV-1 LUCIA of BRCA2 deficient (Capan-1) human pancreatic cells infected with HIV-1 IN⁺ retrovirus stocks.

FIG. 2C shows HIV-1 LUCIA results for RAD52 deficient mouse ES cells infected with HIV-1 IN⁺ retrovirus stocks.

All data are given as the luciferase activity relative to wild type (^{+/+}) cells.

FIG. 3 shows that over-expression of RAD52 impairs retroviral infection.

FIG. 3A shows LUCIA results in the left graph and immunoblot analysis of the Hela cell clones in the right panel for parental Hela cells (HELA), Hela cell clones stably transfected with vector DNA only (IRES-1 and -2) and Hela cell clones stably over-expressing HA-RAD52 (RAD52-1 through 5) infected with HIV-1 IN⁺ retroviral stocks. LUCIA results are expressed as luciferase activity relative to parental Hela cells. Western blots of Hela cell clones were performed using antibodies raised against the HA-tag epitope, RAD52, Ku70, Ku80 and β-actin (loading control).

FIG. 3B shows PCR analysis of stably integrated HIV-1 DNA in RAD52^{-/-} ES cells and HeLa cell clones overexpressing HA-Rad52 at 21 days after infection with HIV-1 luciferase retroviral stocks. The number of stable integration events was assessed by PCR of luciferase DNA (left panels) in relation to total genomic DNA as determined by PCR of GAPDH DNA (right panels). Stably integrated HIV-1 luciferase DNA for RAD52^{-/-} ES cells was compared to parental RAD52^{+/+} ES and HA-Rad52 everexpressing HeLa cell clone RAD52-3 compared to that of the control HeLa:IRES-1 cell clone. PCRs were performed using 1:10 and 1:100 dilutions of high molecular weight genomic DNA to illustrate the linearity of the amplification.

FIG. 4 shows siRNA mediated knockdown of Rad52 expression enhances HIV-1 vector transduction.

FIG. 4A shows HEK-293 cells were transiently co-transfected with the HA-Rad52 expression plasmid and either a non-specific control (−) or Rad52 specific (+) siRNA. Specific knockdown of HA-Rad52 protein expression by the Rad52 siRNA was confirmed by immunoblot analysis with anti-HA tag antibodies. Blots were stripped and re-probed for β-actin and served as both loading and siRNA specficicity controls.

FIG. 4B shows HIV-1 LUCIA results for HeLa and HA-Rad52 overexpressing clones RAD52-3 and RAD52-5 after transfection with the Rad52 siRNA. Knockdown of Rad52 expression led to an increase in HIV-1 luciferase transduction for all cell clones. Data are given as the average luminescence (cps) from at least six wells of a 96-well plate.

FIG. 5 illustrates the finding that the DNA binding domain of RAD52 is required for inhibition of retroviral infection. FIG. 5A shows schematic diagrams of RAD52 and deletion mutants showing functional domains. DNA=DNA binding domain; RAD52=RAD52 self-association domain; RPA RPA binding domain; RAD51=Rad51 binding domain; NLS=nuclear localization signal. Amino acid residues are numbered.

FIG. 5B shows HIV LUCIA results in the left graph and immunoblot analysis of cell lysates in the right panel for HEK-293 cells transiently transfected with full-length (FL) RAD52 and RAD52 deletion mutant expression plasmids then infected with HIV-1 IN⁺ retroviral stocks. LUCIA results are expressed as luciferase activity relative to untransfected HEK-293 cells. Western blots were performed using anti-HA tag antibodies then the blots stripped and re-probed for β-actin (loading control).

FIG. 5c shows chromosomal immunoprecipitation (ChIP) analysis of HEK-293 cells transiently transfected with FL-Rad52 or Rad52 deletion mutant expression plasmids and infected with HIV-1 luciferase retroviral stocks. The level of HIV-1 DNA physically associated with HA-Rad52 was determined by immunoprecipitation with anti-HA antibodies and detected by PCR using primers against HIV-1 LTR sequences. Non-specific control immunoprecipitations were performed using either no antibody (−) or an IgG1 isotype control anti-FLAG tag antibody. As a reference the total amount of HIV-1 LTR DNA formed during a typical infection is also shown (INPUT). PCRs were performed using 1:10 and 1:100 dilutions of ChIP DNA to illustrate the linearity of the amplification

FIG. 6 shows overexpression of Rad52 can compete with Ku for binding to HIV-1.

FIG. 6A shows results of experiments in which HEK-293 cells were transiently transfected with an increasing amount of HA-Rad52 expression plasmid then infected with HIV-1 luciferase retroviral stocks. HIV-1 luciferase transduction assay results are shown in the left graph and immunoblot analysis of cell lysates are shown in the right panel. Results are expressed as luciferase activity relative to untransfected HEK-293 cells. Immunoblots were performed using anti-HA tag antibodies then the blots stripped and re-probed for β-actin (loading control).

FIG. 6B shows competitive PCR-ChIP analysis of HEK-293 cells transiently transfected with increasing amounts of HA-Rad52 expression plasmid and infected with HIV-1 luciferase retroviral stocks. Results show the amount of immunoprecipitated HIV-1 DNA as determined by PCR of 1:10 diluted DNA using specific primers against HIV-1 LTR sequences. HIV-1 DNA associated with either HA-Rad52 or Ku was immunoprecipitated using antibodies against the HA-tag or Ku80. Non-specific control immunoprecipitations were performed using no antibody (−) or an IgG1 isotype control anti-FLAG tag antibody. PCR amplifications were normalised to the amount of HIV-1 LTR DNA used per ChIP (INPUT DNA).

FIG. 6C shows results of immunoblot analysis of input extracts used to perform the ChIP assays. The amount of HA-Rad52 or Ku80 protein used per ChIP analysis was determined by immunoblotting using anti-HA tag or anti-Ku80 antibodies.

FIG. 7 shows that unintegrated 2-LTR circle DNA formation is impaired by Rad52 expression. The results show semi-quantitative PCR analysis of DNA extracted from HIV-1 infected cells with different levels of Ku70 or Rad52 expression. DNA was extracted at indicated times post virus addition and analysed for circular 2-LTR HIV-1 DNA. PCR of the housekeeping gene GAPDH was performed as controls. Southern blots were performed using HIV-1 LTR or GAPDH radiolabelled probes and bands quantified by densitometry. All PCR quantification results are expressed as a normalised ratio of 2-LTR DNA:GAPDH control DNA.

FIG. 7A shows PCR analysis of wild type (KU70^{+/+}; J1) and KU70^{-/-} mouse ES cells. The quantification of bands by densitometric analysis is shown in the graph and an example autoradiograph is shown below.

FIG. 7B shows PCR analysis of wild type (RAD52^{+/+}; 1B10) and RAD52^{-/-} mouse ES cells.

FIG. 7C shows PCR analysis of HEK-293 cells transfected with either full length HA-Rad52 (Rad52 FL) or DNA-binding deletion mutant HA-Rad52 (Rad52 a43-177) expression plasmids

FIG. 8 shows overexpression of Rad52 does not enhance HIV-1 mediated apoptosis. HeLa and two Rad52-overexpressing stable cells clones (RAD52-3 and RAD52-5) were infected with mock or HIV-1 luciferase retrovirus stocks at an MOI=10. At increasing time points after infection cells were harvested and the number of cells undergoing apoptosis quantified by annexin-V staining and flow cytometry. Live/dead cell discrimination was also performed by counter-staining with propidium idodide (PI).

FIG. 8A shows flow-cytometric dot-plots of apoptotic cell populations at 38 hours after infection with mock or HIV-1 luciferase retrovirus stocks. The lower right quandrant (high annexin-V, low PI) of each panel represents early apoptotic cells. The upper right quandrant (high annexin-V, high PI) represents late apoptotic and dead cells. The percentage of cells in each quandrant is shown.

FIG. 8B shows the percentage of cells undergoing the early stages of apoptosis at increasing time points after infection with HIV-1 luciferase retrovirus stocks.

FIG. 8C shows the percentage of both dead and apoptotic cells (all high annexin-V stained cells) at increasing time points after HIV-1 luciferase retrovirus infection.

FIG. 9 illustrates without limitation to the invention a model for the effects of RAD52 and Ku in modulating retroviral infection. This model fits with our data. Should further work lead to modification of the model, this will not affect aspects and embodiments of the present invention that are supported by the data herein showing targeting of RAD52 activity to alter retroviral integration. In the model, unintegrated linear viral DNA is the direct substrate for integration but may also be a signal for apoptosis. In addition to unintegrated linear viral DNA, apoptosis may also by signalled by excessive or unrepaired host cell DNA damage caused by integration. During the course of a normal retroviral infection linear viral DNA ends are bound by Ku, which activates NHEJ repair and results in the formation of 2-LTR DNA circles. Removal of the apoptotic signal could be achieved through the physical elimination of the double-stranded viral DNA through circularisation, the phosphorylation activity of the DNA-PK kinase on down-stream signalling proteins, or both. In the absence of Ku, the NHEJ pathway is not activated, resulting in apoptosis of the host cell. Alternatively, when Rad52 protein levels are high, activation of the NHEJ pathway might be inhibited, possibly by interference of Rad52 with Ku binding to viral DNA ends, thus modulating the efficiency of 2-LTR circle DNA formation. However, since susceptibility towards apoptosis appears unchanged when Rad52 is bound to the ends of linear viral DNA this indicates that Rad52 binding is sufficient to suppress apoptosis. A reduction in transduction efficiency may therefore result from the binding of Rad52 to linear viral DNA, inhibiting the association or recruitment of other protein factors, such as Inil, BAF, hRad18 and integrase, that are required for efficient integration. Loss or repression of Rad52 may remove this inhibition providing enhanced integration activity and a concomitant increase in retroviral infection efficiency.

Reverse transcription and integration of viral double-stranded DNA into a host cell's genome are essential steps in the retroviral lifecycle. These steps are mediated by the retroviral proteins reverse transcriptase (RT) and integrase (IN) respectively. After virus entry, the retroviral RNA is reverse transcribed by RT to produce a linear double-stranded DNA copy that includes directly repeated sequences at each end, known as long terminal repeats (LTR). The linear retroviral cDNA is then 3′-recessed at both ends and joined to host cell chromosomal DNA via a direct trans-esterification reaction catalysed by IN. This strand transfer process results in short staggered DNA strand breaks in the host cell's DNA at the site of attachment (Brown, 1990). Host cell DNA repair proteins must effectively repair these gapped DNA intermediates in order to complete the integration process. Several host cell DNA repair proteins and pathways, including Poly(ADP-ribose)-polymerase (PARP; Gaken et al., 1996; Ha et al., 2001), ataxia telangiectasia mutated (ATM; Daniel et al., 2001), ATM-Rad3-related (ATR; Daniel et al., 2003), hRad18 (Mulder et al., 2002) and the NHEJ DNA repair pathway (Daniel et al., 1999; Li et al., 2001; Jeanson et al., 2002), have all been shown to be involved in the establishment of productive retroviral infections.

In addition to linear retroviral cDNA, covalently closed circular molecules containing 1-LTR, or 2-LTR sequences (arranged in tandem) can also be detected in the nuclei of infected cells. However, only the linear retroviral cDNAs are thought to be substrates for integration, with circular cDNA forms being unproductive by-products of infection (Coffin et al., 1997). 2-LTR circles often contain insertions or deletions and arise through end joining of the linear retroviral cDNA. Recent evidence has been provided suggesting that the NHEJ DNA repair pathway is responsible for 2-LTR DNA circle formation (Li et al., 2001; Jeanson et al., 2002). Consistent with this idea are the findings that Ku, a key protein in NHEJ DNA repair, has been shown to be physically associated with Ty1 retrotransposon (Downs and Jackson, 1999) and retroviral (Li et al., 2001) pre-integration complexes.

Loss of the NHEJ DNA repair pathway (Li et al., 2001; Daniel et al., 1999) renders mammalian cells susceptible to apoptotic cell death following retroviral infection. Li et al. (2001) have suggested that excess double-stranded linear DNA product resulting from RT activity could by itself represent an apoptotic signal, rather like a DSB, that needs to be removed by the action of the NHEJ pathway and the concomitant formation of 2-LTR circles. This process should be IN independent; however, there is some uncertainty here, as Daniel et al. (1999) in their assay system suggested IN activity is required for retrovirally-induced apoptosis in NHEJ-deficient cells.

Together, these studies suggest that the process of retroviral integration into the host cell genome represents an interesting biological system for the study of DNA damage response for a number of reasons: (i) Retroviral intermediates stimulate a variety of different DNA damage signalling and repair pathways. (ii) These retrovirally-induced products and intermediates in the absence of DNA repair could represent pro-apoptotic signals. (iii) As viral gene expression only occurs after a completed integration process (Naldini et al., 1996), these various aspects of mammalian DNA damage response can easily be studied using retroviral-based vectors containing reporter genes. (iv) Integration as an essential step in the retroviral life cycle represents a potential target for the treatment of retroviral infections, such as HIV-1.

The repair of DNA DSBs can occur through either homologous recombination (HR) or non-homologous end-joining (NHEJ) DNA repair pathways (See Khanna and Jackson, 2001 review). Repair of DSBs by HR requires an undamaged homologous DNA sequence for use as a repair template to restore the break in the damaged DNA. Homologous recombination-directed DSB repair proceeds through either gene conversion or single-strand annealing (SSA) pathways (Liang et al., 1998; Paques and Haber, 1999). Gene conversion requires the resection of the DNA ends at the DSB to form 3′ single-stranded DNA overhangs. These are then used to invade a homologous DNA molecule and copy information from the undamaged partner. A sister chromatid, available during S and G₂ phases of the cell cycle, is the normal template for this type of HR repair (Johnson and Jasin, 2000). Proteins involved in HR pathways are the so-called RAD52 group of proteins that includes RAD52, RAD51 and its paralogs XRCC2, XRCC3, Rad51B, Rad51C, and Rad51D (Thompson 2001, O'Regan et al., 2001). Furthermore, the BRCA1 and BRCA2 proteins, encoded by genes associated with breast cancer predisposition, have also been implicated in HR directed gene conversion pathways (Moynahan et al., 1999; Moynahan et al., 2001; Davis et al., 2001). An alternative homology-directed DSB repair pathway (SSA) can operate under special conditions where a DSB is made between two directly repeated DNA sequences. Repair results in the loss of one of the repeats and any intervening DNA sequence. Homology directed DSB repair through SSA depends on RAD52, but not on Rad51 (Paques and Haber, 1999).

Unlike HR, NHEJ DNA repair does not require an undamaged DNA partner or extensive sequence homology but joins the broken ends of a DSB together. NHEJ DNA repair can occur in the absence of a sister chromatid template and often results in deletions or insertions at the DSB. NHEJ repair requires the DNA-end binding activity of the Ku70/80 heterodimer, its interacting partner, the catalytic subunit of DNA-dependent protein kinase (DNA-PKcs), and the complex between DNA ligase IV and XRCC4 (Van Gent et al., 2001).

The choice of HR or NHEJ directed DNA repair is largely thought to be determined by cell cycle status, but a degree of overlap between DSB repair pathways occurs. DNA repair pathway utilisation may also be influenced by competitive binding between HR and NHEJ DNA repair proteins to sites of DNA damage. For example, competition between Ku directed NHEJ and RAD52 directed HR DNA repair pathways have been proposed where the choice of which repair pathway is invoked is dependent upon whether Ku or Rad52 binds to the DNA end (Van Dyck et al., 1999) and the presence of a two-ended DSB (Pierce et al., 2001).

Retroviruses are RNA viruses that must insert a DNA copy (cDNA) of their genome into the host chromosome in order to carry out a productive infection. When integrated, the virus is termed a provirus (Varmus, 1988). Some eukaryotic transposable DNA elements are related to retroviruses in that they transpose via an RNA intermediate. These elements, termed retrotransposons or retroposons, are transcribed into RNA, the RNA is copied into double-stranded (ds) DNA, and then the dsDNA is inserted into the genome of the host cell.

Retroviruses are of considerable risk to human and animal health, as evidenced by the fact that retroviruses cause diseases such as acquired immune deficiency syndrome (AIDS; caused by human immunodeficiency virus; HIV-1), various animal cancers, and human adult T-cell leukaemia/lymphoma (Varmus, 1988); also retroviruses have been linked to a variety of other common disorders, including Type I diabetes and multiple sclerosis (Conrad et al., 1997; Perron et al. 1997 and Benoist and Mathis, 1997). In many but not all cases, cancer formation by certain animal retroviruses is a consequence of them carrying oncogenes. Furthermore, retroviral integration and retrotransposition can result in mutagenic inactivation of genes at their sites of insertion, or can result in aberrant expression of adjacent host genes, both of which can have deleterious consequences for the host organism. Various aspects and embodiments of the present invention are concerned with inhibiting retroviruses, with the aim of treatment and/or prevention of retroviral-associated disorders, including those listed here.

Retroviruses are also becoming more and more commonly used for gene delivery and are likely to play increasingly important roles in gene therapy (see e.g. Hawley 2001; Scherr and Eder 2002). Of particular interest in the present invention is use of retroviral vectors either in vivo or ex vivo, for example in cells removed from the body for treatment then subsequent return to the body. Such cells may be haematopoietic stem cells (HSCs).

In one aspect, the present invention provides a method of promoting retrovirus integration into the genome of a mammalian cell, by means of targeting RAD52 to reduce its activity in the cell, by inhibiting RAD52 protein interaction with DNA (double-stranded DNA ends) and/or by reducing the level of RAD52 protein within the cell—by eliminating protein if produced and/or inhibiting its production. Activity of RAD52 may be modulated by targeting a product of another gene, e.g. a protein that affects RAD52 expression, stability or activity.

Methods of treatment of the human or animal body by way of therapy may be excluded. In principle a method of the invention may be carried out in vivo, for example in a method of therapy (which may be prophylactic) or in a non-therapeutic method. Of particular interest is a method that may be carried out in vitro or ex vivo, e.g. on transplant material, such as cells or tissue removed from the body for subsequent return (for instance stem cells).

Various aspects and embodiments of the present invention are provided as set out in the accompanying claims, and as disclosed anywhere herein.

Inhibition of RAD52 DNA-binding activity, in particular binding to double-stranded DNA ends, may be achieved in any of numerous different ways, without limitation to the nature and scope of the present invention.

In certain embodiments of the present invention, RAD52 is targeted for inhibition, that is to say binding of RAD52 with DNA itself or multimerisation between RAD52 subunits is inhibited by a substance that interferes with the binding or multimerisation. A substance may inhibit binding by inhibiting physical interaction between RAD52 and DNA or between RAD52 subunits, or by binding in a way that has a steric effect on the conformation of binding site. Precisely how the RAD52 activity or function in inhibiting retroviral integration is inhibited need not be relevant to practising the present invention.

The activity or function of RAD52 may be inhibited, as noted, by means of a substance that interacts in some way with the protein. Another approach, and in some embodiments a preferred option, employs regulation at the nucleic acid level to inhibit activity or function by down-regulating production of the component.

For instance, expression of a RAD52 gene may be inhibited using anti-sense technology. The use of anti-sense genes or partial gene sequences to down-regulate gene expression is well-established.

Anti-sense oligonucleotides may be designed to hybridise to the complementary sequence of nucleic acid, pre-mRNA or mature mRNA, interfering with the production of RAD52 so that its expression is reduced or completely or substantially completely prevented. In addition to targeting coding sequence, antisense techniques may be used to target control sequences of a gene, e.g. in the 5′ flanking sequence, whereby the antisense oligonucleotides can interfere with expression control sequences. The construction of antisense sequences and their use is described for example in Peyman and Ulman, Chemical Reviews, 90:543-584, (1990) and Crooke, Ann. Rev. Pharmacol. Toxicol., 32:329-376, (1992).

Oligonucleotides may be generated in vitro or ex vivo for administration or anti-sense RNA may be generated in vivo within cells in which down-regulation is desired.

Thus, double-stranded DNA may be placed under the control of a promoter in a “reverse orientation” such that transcription of the anti-sense strand of the DNA yields RNA which is complementary to normal mRNA transcribed from the sense strand of the target gene. The complementary anti-sense RNA sequence is thought then to bind with mRNA to form a duplex, inhibiting translation of the endogenous mRNA from the target gene into protein. Whether or not this is the actual mode of action is still uncertain. However, it is established fact that the technique works.

The complete sequence corresponding to the coding sequence in reverse orientation need not be used. For example fragments of sufficient length may be used. It is a routine matter for the person skilled in the art to screen fragments of various sizes and from various parts of the coding or flanking sequences of a gene to optimise the level of anti-sense inhibition. It may be advantageous to include the initiating methionine ATG codon, and perhaps one or more nucleotides upstream of the initiating codon. A suitable fragment may have about 14-23 nucleotides, e.g. about 15, 16 or 17.

An alternative to anti-sense is to use a copy of all or part of the target gene inserted in sense, that is the same, orientation as the target gene, to achieve reduction in expression of the target gene by co-suppression; Angell & Baulcombe (1997) The EMBO Journal 16, 12:3675-3684; and Voinnet & Baulcombe (1997) Nature 389: pg 553). Double stranded RNA (dsRNA) has been found to be even more effective in gene silencing than both sense or antisense strands alone (Fire A. et al Nature, Vol 391, (1998)). dsRNA mediated silencing is gene specific and is often termed RNA interference (RNAi). RNA interference is a two-step process. First, dsRNA is cleaved within the cell to yield short interfering RNAs (siRNAs) of about 21-23 nt length with 5′ terminal phosphate and 3′ short overhangs (˜2 nt). The siRNAs target the corresponding mRNA sequence specifically for destruction (Zamore P. D. Nature Structural Biology, 8, 9, 746-750, (2001) RNAi may be also be efficiently induced using chemically synthesized siRNA duplexes of the same structure with 3′-overhang ends (Zamore PD et al Cell, 101, 25-33, (2000)). Synthetic siRNA duplexes have been shown to specifically suppress expression of endogenous and heterologous genes in a wide range of mammalian cell lines (Elbashir S M. et al. Nature, 411, 494-498, (2001)).

See also Fire (1999) Trends Genet. 15: 358-363, Sharp (2001) Genes Dev. 15: 485-490, Hammond et al. (2001) Nature Rev. Genes 2: 1110-1119 and Tuschl (2001) Chem. Biochem. 2: 239-245, Hannon (2002) Nature 418 (6894):244-51, Ueda (2001) J Neurogenet. 15(3-4): 193-204, Lindenbach (2002) Mol Cell. 9(5): 925-7, Brant (2002) Biochim Biophys Acta. 1575(1-3): 15-25, Grishok (2002) Adv Genet. 46:339-60, Hutyagner (2002) Curr Opin Genet Dev. 12(2):225-32, Ullu et al. (2002) Philos Trans R Soc Lond B Biol Sci. 357(1417): 65-70.

Another possibility is that nucleic acid is used which on transcription produces a ribozyme, able to cut nucleic acid at a specific site—thus also useful in influencing gene expression. Background references for ribozymes include Kashani-Sabet and Scanlon, 1995, Cancer Gene Therapy, 2(3): 213-223, and Mercola and Cohen, 1995, Cancer Gene Therapy, 2 (1), 47-59.

Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992 or later edition, and Molecular Cloning: a Laboratory Manual: 3rd edition, Sambrook and Russell, 2001, Cold Spring Harbor Laboratory Press.

The nucleic acid and protein sequences of RAD52 in humans and mouse are available from the GenBank database, under the following accession numbers: human RAD52 cDNA (U27516); human RAD52 protein (AAA87554); mouse RAD52 cDNA (AF004854); mouse RAD52 protein (AAB69174).

Techniques targeting RAD52 expression are particularly useful in embodiments where it is desired temporarily to inhibit RAD52 DNA-binding activity in a cell, and thus promote retroviral integration. This is especially useful in ex vivo cell manipulation where a retroviral vector is introduced into a cell and integrated into the genome to encode and allow for production of a therapeutic gene product in the cell, then the cell is returned to the body. Release from temporary inhibition allows RAD52 to return to its normal functions in the cell.

Retroviral vectors may be introduced into cells using any suitable technique. The introduction, which may (particularly for in vitro or ex vivo introduction) be generally referred to without limitation as “transformation”, may employ any available technique. For eukaryotic cells, suitable techniques may include calcium phosphate transfection, DEAE-Dextran, electroporation, liposome-mediated transfection and transduction using retroviruses.

As noted, a cell in which retroviral integration is promoted may be a stem cell. A stem cell may be returned to the body of a donor from which it has been removed, after integration of a retroviral vector into the genome of the stem cell, e.g. for a therapeutic purpose. Furthermore, non-human animals can be generated from mammalian non-human (e.g. mouse) embryonic stem (ES) cells into which a desired retroviral vector has been introduced. This may be for a research purpose, e.g. in generation of a model for study of a clinical disorder or disease. See for instance page 2 of WO97/05268 and the references cited there for specific background information.

Agents that promote retroviral integration into a cell, such as a substance that binds RAD52 and/or inhibits RAD52 binding to DNA, or a substance that inhibits RAD52 production (e.g. RNA with nucleotide sequence complementary to a RAD52 gene sequence, which RNA is double-stranded RNA or antisense RNA, or a ribozyme specific for a RAD52 gene sequence) can be obtained using routine assay and screening techniques available in the art.

Similarly, appropriate assays and screens can be used to obtain agents that inhibit retroviral integration by increasing RAD52 activity, especially binding to DNA. The action of such an agent may be for example by potentiating or stabilising RAD52 binding to DNA, e.g. by reducing off-rate, or by increasing production of RAD52 in a cell, e.g. by increasing expression by acting on a promoter or other regulatory element controlling transcription or translation, or by stabilising RAD52 against degradation in the cell.

It is well known that pharmaceutical research leading to the identification of a new drug may involve the screening of very large numbers of candidate substances, both before and even after a lead compound has been found. This is one factor that can make pharmaceutical research very expensive and time-consuming. Means for assisting in the screening process can have considerable commercial importance and utility. Such means for screening for substances potentially useful in inhibiting retroviral and/or retrotransposon activity is provided according to the present invention. Substances identified as promoters or potentiators of RAD52 activity, by action on the protein or a subunit or by facilitation or stabilisation of its binding to DNA, or by increasing its expression by upregulation of transcription of the gene or by stabilisation of encoding mRNA, represent an advance in the fight against retroviral diseases (for instance), since they provide basis for design and investigation of therapeutics for in vivo use.

As noted, RAD52 binds DNA, specifically ends at DNA double-strand breaks (DSBs). To assay for inhibition or promotion of RAD52 binding to these, suitable e.g. synthetic preparations of DNA may be provided.

Biochemical methods, such as PCR or nucleic acid hybridisation/detection methods, may be used, e.g. to detect the chemical structure of integration products. Retroviral integration and/or retrotransposition may be scored for example by detection using standard genetic, biochemical or histological techniques.

In assays and screens according to embodiments of the present invention, appropriate control experiments may be performed in accordance with appropriate knowledge and practice of the ordinary skilled person. Experiments may be performed in the presence and absence of a test compound, substance or agent.

For potential therapeutic purposes, the RAD52 protein used in the assay may be human, or non-human mammalian, e.g. murine, mouse, rat, rabbit, guinea pig, sheep, goat, cow, pig, cat or dog.

Of course, reference to RAD52 in an assay may be taken to refer to a derivative, variant or analogue of the relevant component which has the requisite, assayable property or activity, in particular ability to bind DNA ends (and thereby inhibit retroviral integration).

Given the teaching provided herein of the ability to inhibit or promote retroviral and/or retrotransposon activity by manipulating RAD52 activity, those of ordinary skill in the art may design assays by employing proteins or fragments thereof that are homologous with RAD52.

Prior to, as well as or instead of being screened for actual ability to affect RAD52 activity, test substances may be screened for ability to interact with or bind RAD52 e.g. in a yeast two-hybrid system (which requires that both the polypeptide component and the test substance can be expressed in yeast from encoding nucleic acid, see e.g. Evan et al. Mol. Cell. Biol. 5, 3610-3616 (1985); Fields & Song Nature 340, 245-246 (1989)). This may for example be used as a coarse screen prior to testing a substance for actual ability to modulate activity. Another example of a similar approach is to use RAD52 protein or a DNA binding fragment thereof to obtain one or more antibody molecules or other specific binding molecules against the protein, e.g. from a phage display library, these antibody molecules or other specific binding molecules may then be tested for ability to affect RAD52 activity and/or retrovirus integration in a suitable test system, in vitro, ex vivo or in vivo.

Following identification of an agent or substance that modulates or affects RAD52 activity, the substance may be investigated further, in particular for its ability to promote or inhibit retroviral and/or retrotransposon integration. Furthermore, it may be manufactured and/or used in preparation, i.e. manufacture or formulation, of a composition such as a medicament, pharmaceutical composition or drug. These may be administered to individuals.

Thus, the present invention extends in various aspects not only to a substance identified as inhibiting retroviral and/or retrotransposon activity in accordance with what is disclosed herein, but also a pharmaceutical composition, medicament, drug or other composition comprising such a substance, a method comprising administration of such a composition to a patient, e.g. for treatment (which may include preventative treatment) of a retroviral disorder, use of such a substance in manufacture of a composition for administration, e.g. for treatment of a retroviral disorder, and a method of making a composition comprising admixing such a substance with a pharmaceutically acceptable excipient, vehicle or carrier, and optionally other ingredients.

A substance for promoting or inhibiting retrovirus and/or retrotransposon integration in accordance with any aspect of the present invention may be formulated in a composition.

A composition (especially one comprising a substance which is an inhibitor of retroviral integration) may include, in addition to said substance, a pharmaceutically acceptable excipient, carrier, buffer, stabiliser or one or more other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at a particular site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Whether it is a polypeptide, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

Targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Instead of administering these agents directly, they may be produced in the target cells by expression from an encoding gene introduced into the cells. The vector may be targeted to the specific cells to be treated, or it may contain regulatory elements that are switched on more or less selectively by the target cells.

The agent may be administered in a precursor form, for conversion to the active form by an activating agent produced in, or targeted to, the cells to be treated.

A composition may be administered alone or in combination with other treatments, either simultaneously or sequentially dependent upon the condition to be treated.

Further aspects and embodiments of the present invention relate to obtaining agents that promote retroviral integration. Such agents may be substances that bind to RAD52 protein and/or interfere with RAD52 protein binding to DNA, e.g. antibody molecules, peptides (e.g. fragments of RAD52) or other molecules. Such agents may be substances that inhibit production of RAD52 protein in cells, such as antisense RNA or siRNA.

Assays and methods of screening in which integration of a retroviral vector is determined may comprise direct detection of a reporter gene within the retrovirus. Reporter genes used for the quantification of retroviral integration events may include antibiotic resistance genes, such as those coding for resistance to Neomycin (G418), Puromycin, or Hygromycin. Alternative reporters include autofluorescent reporters such as the green fluorescent protein GFP or variants thereof or enzymatic gene products such as β-galactosidase, or chloramphenicol acetyl transferase (CAT). However, the preferred reporter gene for retroviral integration assays may consist of genes coding for products capable of generating chemiluminescence. The preferred reporter gene may be the firefly (Photinus pyralis) luciferase gene that provides both a high level of sensitivity and as a result of this, an ability to be used in high throughput assays. Alternatives to the firefly luciferase gene include the Sea Pansy (Renilla reniformis) luciferase gene product.

An assay of retroviral integration into host cells may include;

introducing a retroviral vector into host cells, e.g. by infection with a retrovirus, said retroviral vector containing a reporter gene encoding a chemiluminescent protein,

causing or allowing expression of said reporter gene from integrated retroviruses; and

determining luminescence generated by said chemiluminescent protein.

Host cells may be transduced/infected with retrovirus in the presence or absence of an agent of interest. The effect of the agent of interest on retroviral integration may then be assessed by comparing the luminescent signals produced in the presence and absence of agent.

Assays may be conveniently carried out in a 96-well microtitre plate format. Reagents and materials for generating and measuring a luminescent end point are well known in the art and are available commercially. Such reagents and materials may be used by a skilled person in accordance with the manufacturer's instructions as appropriate. The retroviral luciferase integration assay (LUCIA) represents a significant improvement on other currently available retroviral integration assays, including the colony formation assay (CFA), which utilises drug resistance markers and takes significantly longer than LUCIA and which is not amenable to High Throughput Screening (HTS), or assays utilising β-galactosidase activity which do not possess the inherent sensitivity of luciferase-based assays. Such other assays are, however, useful in determination of retroviral integration where such determination is needed in accordance with an aspect or embodiment of the present invention.

The experimental basis for the invention and illustrative embodiments of the invention will now be described in more detail, with reference to the accompanying drawings. All publications mentioned anywhere in the text are incorporated herein by reference.

Experimental

Here we show that RAD52 can modulate the outcome of retroviral infection (exemplified using recombinant HIV-1 vector) by markedly reducing the efficiency of productive integration events. No major phenotype has previously been described for a RAD52 deficiency in mammalian cells. Mutations in other HR proteins (XRCC2, XRCC3 and BRCA2) do not affect retroviral transduction rates. Our results provide indication that the HR repair pathway per se does not influence retroviral infection in mammalian cells. Furthermore, although we can demonstrate competitive binding between Rad52 and Ku to retroviral cDNA ends and a subsequent modulation of NHEJ DNA repair activity, this interference alone cannot adequately explain the effect Rad52 has on retroviral infection. The mechanism of attenuation of retroviral infection by RAD52 appears to be based upon physical interference between Rad52 and the integration machinery, most likely through competitive binding of RAD52 to unintegrated retroviral cDNA ends.

Results

RAD52 but not Other HR Proteins are Involved in Modulating Retroviral Infection

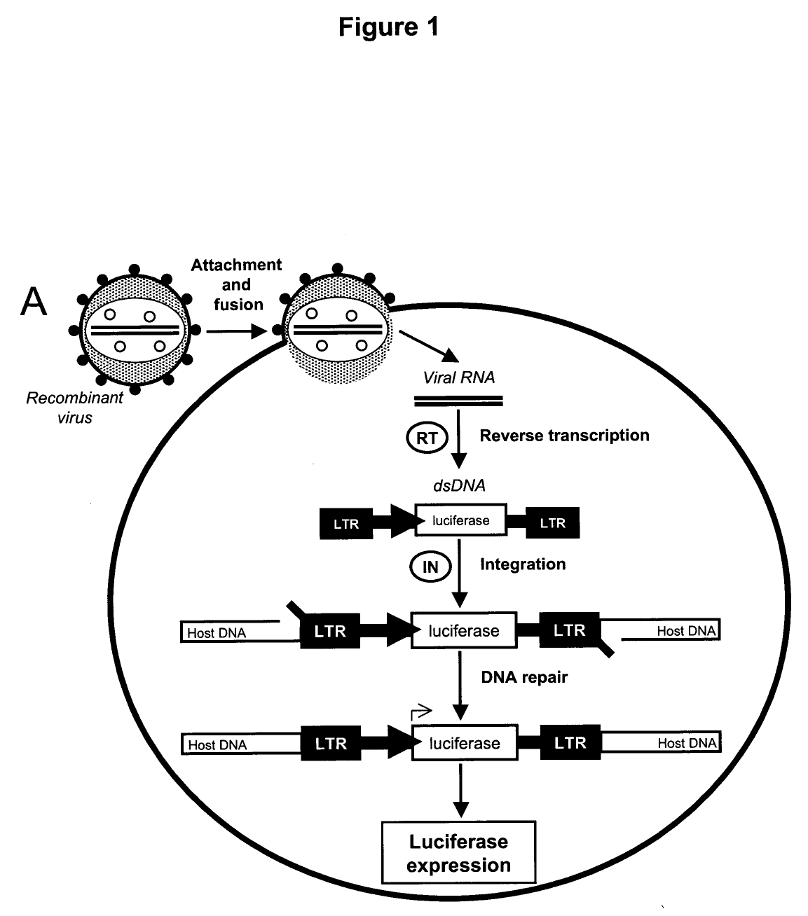

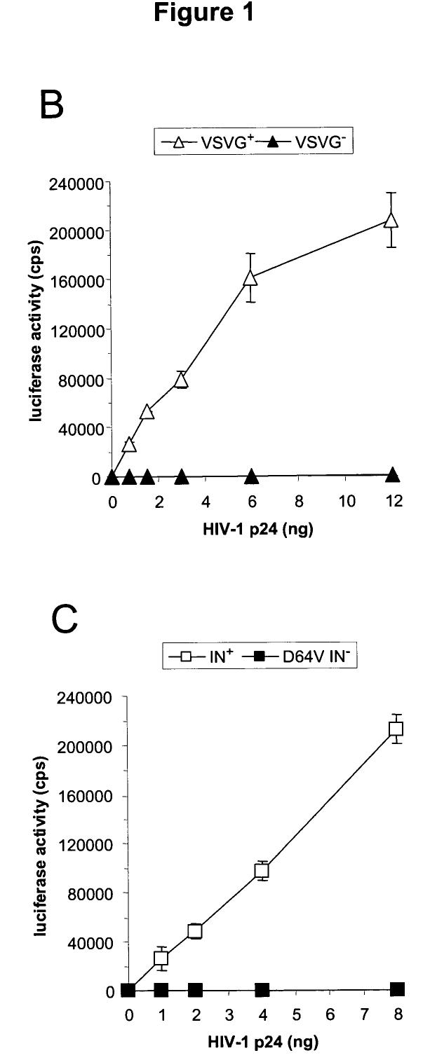

To quantitatively assess retroviral vector transduction efficiency, we used single-step recombinant HIV-1 based vectors which contain the firefly luciferase reporter gene (see FIG. 1A and Materials and Methods). FIG. 1B and FIG. 1C provides an indication of the sensitivity and dynamic range of this HIV-1 luciferase integration assay, which we term LUCIA. FIG. 1B shows that viral entry is required for luciferase expression, as recombinant viruses lacking the VSVG envelope glycoprotein (VSVG⁻) give no signal in LUCIA. Similarly, FIG. 1C shows that luciferase expression only occurs with recombinant viruses that contain a functional integrase protein (IN⁺). Viruses that contain the D64V integrase mutation (D64V IN⁻) do not give rise to luciferase signals in LUCIA and demonstrates that gene expression only occurs after integration is completed. When using functional retroviral stocks (VSVG⁺/IN⁺), a direct positive correlation between the amount of virus added and the luciferase signal generated in LUCIA is observed.

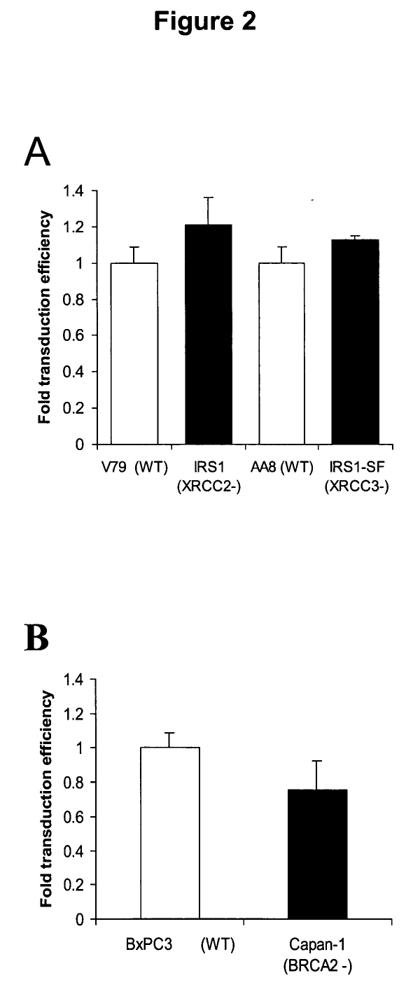

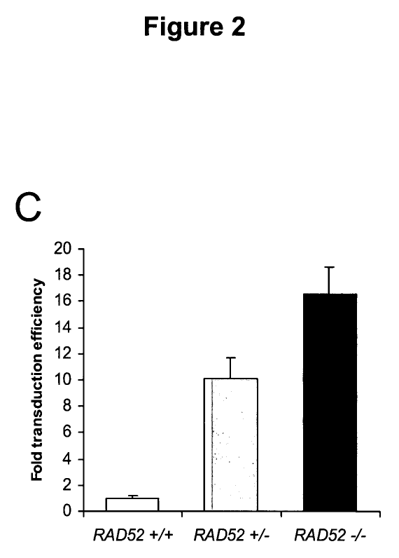

Cells defective in RAD52, Rad51 paralogs, XRCC2 and XRCC3, or in BRCA2, all components of the HR pathway, were infected with HIV-1 IN⁺ luciferase reporter gene vectors and transduction efficiencies were determined by luciferase activity after 48 hours (FIG. 2).

FIGS. 2A and 2B shows HIV-1 LUCIA results of cells defective in the Rad51 paralogs, XRCC2 and XRCC3 (FIG. 2A), or in BRCA2 (FIG. 2B). Loss of these key components in the HR pathway in mammalian cells did not show any effect on retroviral transduction efficiencies.

In contrast, FIG. 2C shows HIV-1 LUCIA results of isogenic mouse ES cells with targeted deletions of none (RAD52^{+/+}), one (RAD52^{+/-}) or both (RAD52^{-/-}) RAD52 alleles. In the absence of RAD52 protein, the efficiency of retroviral transduction was increased by 16-fold. Integration efficiency was sensitive to the level of RAD52 gene dosage, since the deletion of one RAD52 allele resulted in a 10-fold increase.

Previous work has demonstrated that mutations in mammalian RAD52 lead to only a minor (1.5-fold) impairment in HR (Rijkers et al., 1998). The magnitude of the effect on retroviral integration efficiency seen in FIG. 2C and the absence of an increase in cells defective in other HR pathway components (FIGS. 2A and 2B) indicates that the role of RAD52 in the retroviral integration process is independent of its role in HR.

Overexpression of Rad52 Inhibits HIV-1 Infection and Stable Integration

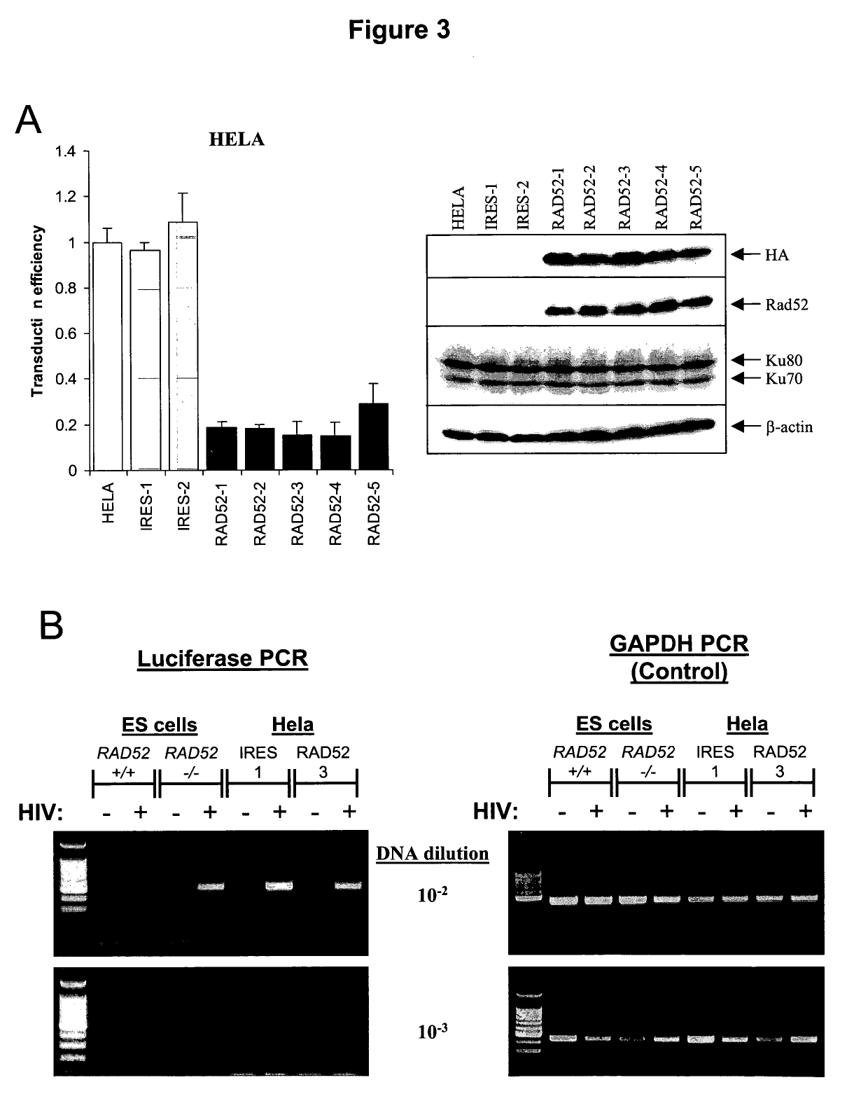

We generated a number of stably transfected Hela cell lines containing either vector DNA (IRES) or vector DNA expressing HA epitope-tagged RAD52. Overexpression of RAD52 was confirmed by immunoblot analysis of two independently isolated cell lines containing vector DNA and five cell lines containing the HA-RAD52 expressing vector (FIG. 3A). Both anti-HA and anti-RAD52 antibodies detected the over-expressed HA-RAD52 protein. Antibodies against Ku70, Ku80 and β-actin served as loading controls.

LUCIA assays revealed that retroviral transduction was equally efficient in Hela and vector transfected cell lines. However, in the Hela cell lines over expressing RAD52, transduction was reduced 5-fold (FIG. 3A).

Both RAD52^{-/-} ES cells and Rad52-overexpressing HeLa clones were transduced with the HIV-1 luciferase vectors and the level of stably integrated, proviral DNA in the cell population determined by PCR. Integrated, proviral DNA was detected by PCR for the presence of the luciferase reporter gene (FIG. 3B). Analysis of total proviral DNA content, as determined by luciferase-directed PCR, in transduced ES cells shows that RAD52^{-/-} cells contain a greater amount of integrated proviral DNA than RAD52^{+/+} cells. In contrast, the Rad52-overexpressing HeLa clone (RAD52-3) show a reduction in total integrated proviral DNA compared to control cells (IRES-1).

These results are consistent with those seen for HIV-1 LUCIA (FIGS. 1C and 2A) and indicate that Rad52 inhibition occurs primarily through impairment of stable HIV-1 integration events. Although Rad52 expression reduces total integrated proviral DNA number these experiments cannot distinguish between the loss of infected cells in the cell population due to apoptosis or by a direct effect on the integration process itself.

RNAi-Mediated Knockdown of Rad52 Expression Enhances Retroviral Infection

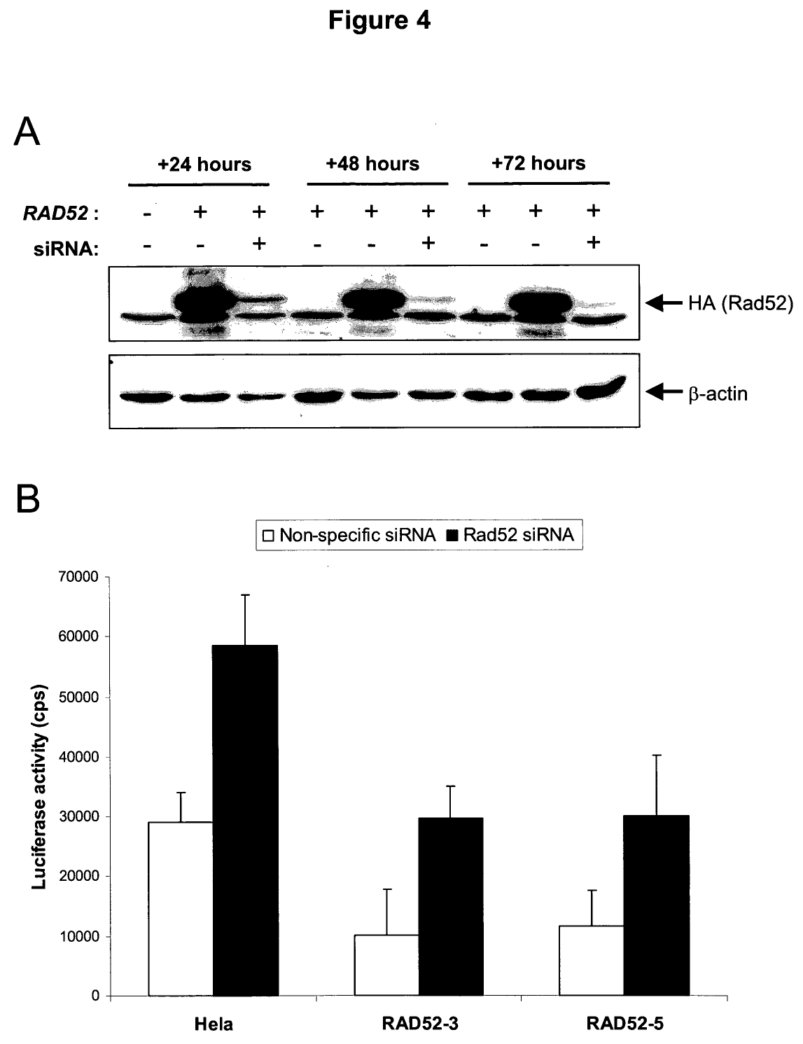

Rad52 protein levels were reduced by RNA interference (RNAi) using small interfering RNAs (siRNAs), a highly specific and potent method to knockdown gene expression (Zamore et al., 2001). A single 21-nt duplex siRNA directed against Rad52 was designed and tested for its ability to knockdown Rad52 protein expression by transient transfection and immunoblot analysis. HEK-293 cells were co-transfected with the HA-Rad52 expression vector and either a non-specific control or Rad52 siRNA. At various times after transfection cells were harvested and HA-Rad52 expression determined by immunoblotting (FIG. 4A). Co-transfection of the HA-Rad52 expression plasmid with the non-specific control siRNA showed good HA-Rad52 expression at all time points. However, co-transfections with the Rad52 siRNA significantly reduced HA-Rad52 expression with maximal knockdown observed at 72 hours after transfection. Re-probing of immunoblots for β-actin served as both loading and specificity controls. This demonstrates that Rad52 expression can be specifically knocked-down using this siRNA duplex.

The Rad52 siRNA was used to knockdown Rad52 expression in both HeLa and Rad52-overexpressing clones RAD52-3 and Rad52-5. HIV-1 luciferase transduction assays were then performed on these cells. The results in FIG. 4B show that Rad52 siRNA-transfected cells demonstrate a significant increase in HIV-1 vector transduction efficiency when compared with cells transfected with the non-specific control siRNA. These data are consistent with what is seen for RAD52 knockout mouse ES cells (FIG. 1C), although the relative increase in HIV-1 transduction efficiency is much lower at only 3 fold over control cells. This discrepancy could be due to the fact that siRNA knockdown is never 100% complete (FIG. 4A) with residual protein and activity always remaining.

These data indicate that the presence of Rad52 is indeed inhibitory towards HIV-1 vector transduction. These data also demonstrate the validity of using siRNAs directed against Rad52 to increase retroviral infection efficiency.

The DNA Binding Activity of RAD52 is Required to Inhibit HIV-1 Infection

RAD52 interacts with two key proteins during HR, namely RPA and Rad51 (Shen et al., 1996a and 1996b; Park et al., 1996; Milne and Weaver, 1993). Although both RPA and Rad51 are essential for HR, they compete for the same substrate. Rad51 forms a nucleoprotein filament on single-stranded DNA that arises after processing of a DSB (reviewed in Paques and Haber, 1999). Subsequently, this nucleoprotein filament searches for homologous duplex DNA and mediates DNA strand exchange. In order to form a nucleoprotein filament on single-stranded DNA, Rad51 needs to displace the single-strand DNA binding protein RPA. Since RPA is a more tenacious single-strand DNA binding protein than Rad51, the Rad51 protein cannot displace RPA by itself. However, the RAD52 protein performs a mediator function that facilitates the displacement of RPA by Rad51. If the observed effect of RAD52 on retroviral transduction is indeed independent of its role in HR, then the domain of RAD52 that is required for its interaction with RPA and Rad51 should be dispensable for the repression of transduction.

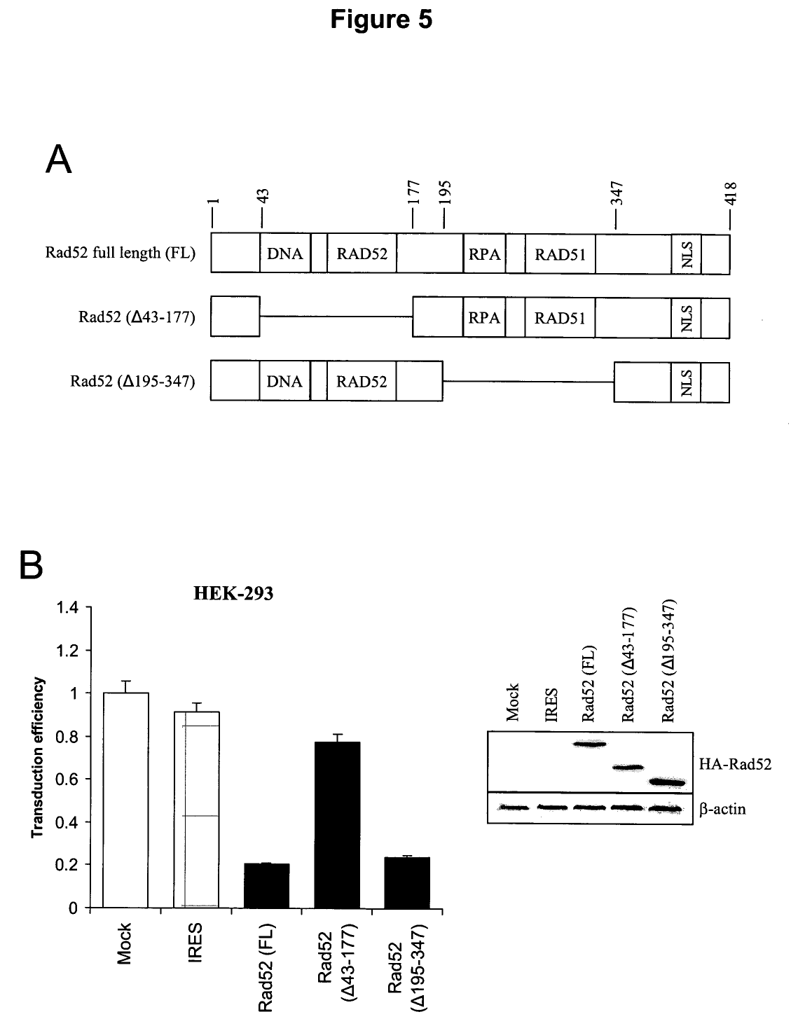

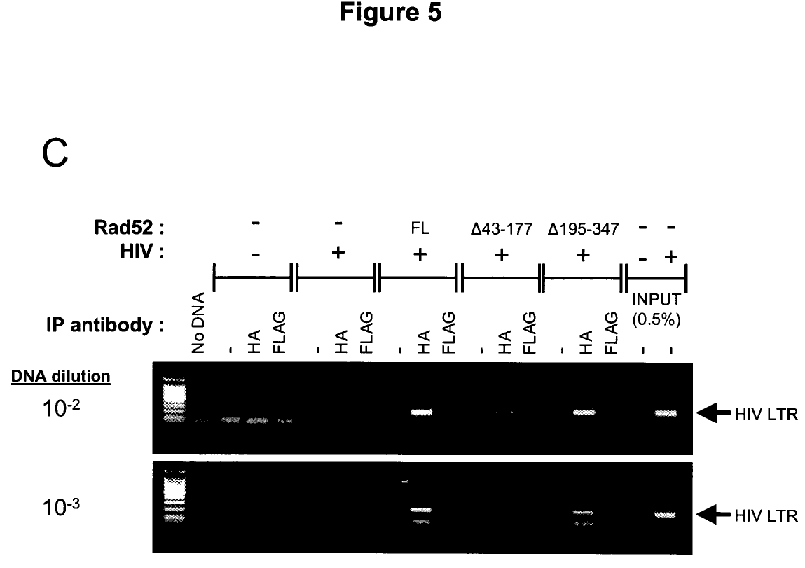

We tested two different internal deletion mutants of RAD52 for their ability to modulate transduction. The first mutant lacks amino acids 43 through 177, which span the RAD52 multimerisation and DNA binding domains (FIG. 5A) (Shen et al., 1996a and 1996b). The second mutant lacks amino acids 195 through 347, which contains the RPA and Rad51 interaction domains (Park et al., 1996). Plasmids expressing full-length RAD52 or either of the two deletion mutants were transiently transfected in HEK-293 cells. The expression of the RAD52-derivative was confirmed by immunoblotting (FIG. 5B).

LUCIA assays demonstrated that only the DNA binding and self-interaction domains of RAD52 were required for the repression of retroviral transduction (FIG. 4B) and not the RPA and Rad51 interaction domains of RAD52. The loss of the RPA interaction domain has previously been shown to abolish the ability of RAD52 to stimulate HR (Park et al., 1996), implying that HR repair activities do not directly influence the course of retroviral infections. However, the requirement for the DNA binding activity of RAD52 suggests that a physical DNA interaction, presumably with HIV-1 DNA ends, is necessary to elicit the inhibitory effects.

The direct association of Rad52 with HIV-1 cDNA ends was confirmed performing chromosomal immunoprecipitation (ChIP) assays on HEK-293 cells transiently transfected with Rad52 expression plasmids and transduced with the HIV-1 luciferase vector (FIG. 5C). Transfections were carried out using full length Rad52 as well as both Rad52 deletion mutant expression plasmids to confirm that they do show the correct DNA binding properties as predicted. Immunoprecipitations using whole cell extracts were carried out with antibodies against the HA-tagged Rad52 and with control antibodies (none and isotype IgG1 anti-FLAG). Associated HIV-1 DNA was then detected by PCR using primers directed against HIV-1 LTR sequences.

FIG. 5C demonstrates that full length Rad52 is indeed associated with HIV-1 DNA ends as HIV-1 LTR DNA sequences were easily amplified from HA ChIPs but not with non-specific control ChIPs. As predicted, the Rad52 Δ43-177 DNA binding mutant shows a greatly reduced affinity for HIV-1 DNA ends whereas the Δ195-347 mutant still binds HIV-1 DNA ends readily.

Rad52 can Compete with Ku for Binding to HIV-1 LTR DNA Ends

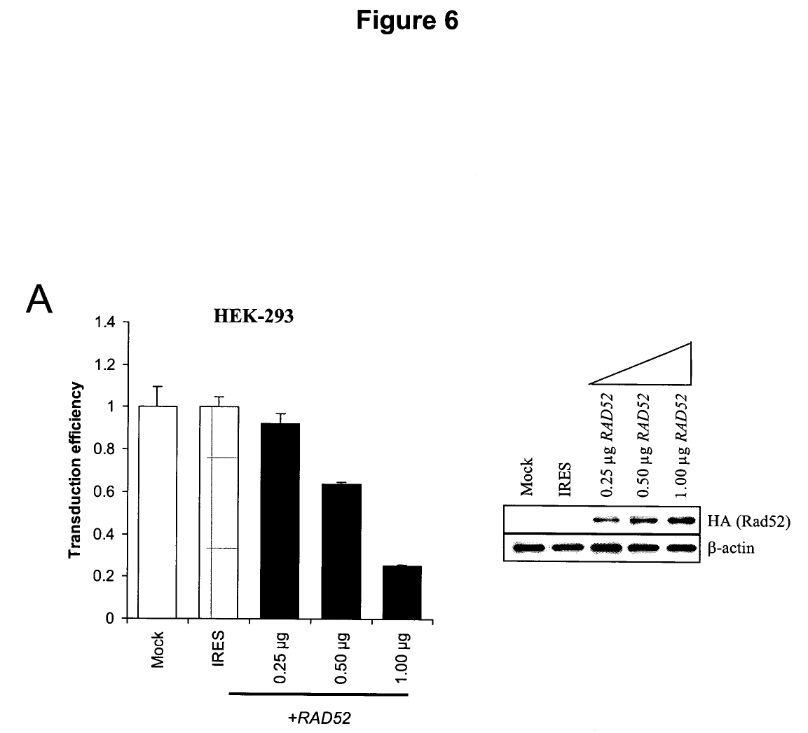

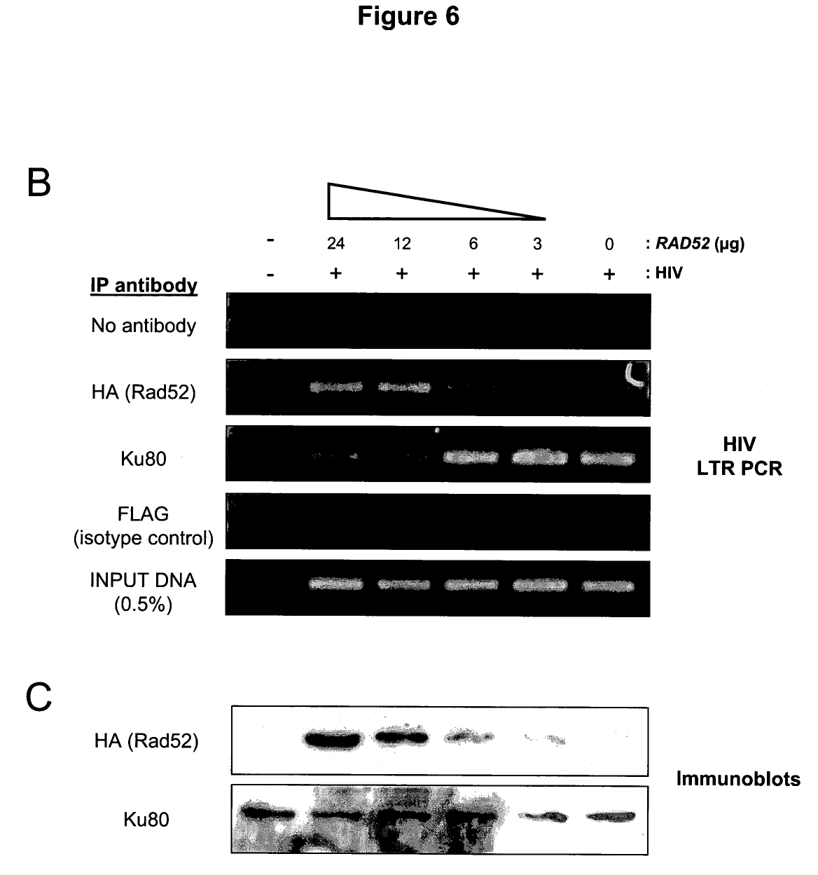

A gene dosage effect of RAD52 on retroviral transduction efficiency was observed in mouse ES cells (FIG. 1C). We investigated whether the repression of transduction by exogenously expressed Rad52 in human cells was also dependent upon the level of Rad52. HEK-293 cells were transiently transfected with increasing amounts of Rad52 expression plasmid DNA. Immunoblot analysis of the cells showed that Rad52 protein levels correlated with the amount of transfected plasmid DNA (FIG. 6A). Importantly, HIV-1 LUCIA demonstrated that the efficiency of retroviral transduction was decreased with increasing amounts of Rad52 protein. The gene dosage effect of RAD52 on retroviral transduction (FIGS. 1C and 6A) coupled with the observation that only Rad52 DNA end binding is required to elicit this effect (FIG. 5) indicates a possible mechanism of inhibition involving competition between proteins for HIV-1 cDNA intermediates.

A number of studies have shown that Ku and NHEJ DNA repair are required for efficient retroviral transduction (Daniel et al., 1999; Li et al., 2001; Jeanson et al., 2002) and Ku is physically associated with retroviral pre-integration complexes (Li et al., 2001; Lin and Engelman 2003), presumably bound to cDNA ends.

Competitive PCR-ChIP assays were performed on HEK-293 cells transfected with increasing amounts of Rad52 expression plasmid DNA and transduced with the HIV-1 luciferase vector. ChIPs were performed using antibodies against Ha-tagged Rad52 and Ku80 and the amount of HIV-1 LTR DNA immunoprecipitated by each antibody was assessed by PCR (FIG. 6B). PCR-ChIP analysis shows that in cells with high Rad52 expression (24 and 12 μg of expression plasmid) the amount of Rad52 associated HIV-1 LTR DNA (anti-HA ChIPs) is significantly greater than those with low levels of Rad52 expression (<6 μg). Crucially, anti-Ku80 ChIPs show the exact opposite finding, with cells expressing high levels of Rad52 showing significantly reduced levels of Ku80 associated HIV-1 LTR DNA compared to cells with low rad52 levels. Together, all these data support the hypothesis that Rad52 can indeed compete with Ku for HIV-1 cDNA ends.

Rad52 Suppresses the Formation of HIV-1 2-LTR Circle DNA

Lack of RAD52 enhances the formation of 2-LTR HIV-1 circle DNA Previously, it has been established that proteins involved in NHEJ are required for the formation of retroviral 2-LTR circle DNA (Li et al., 2001; Jeanson et al., 2002). The absence of RAD52 and Ku have now, by our present work, been shown to have opposite effects with regard to the modulation of retroviral transduction efficiency (see the results presented herein in comparison, for example, with FIG. 3 of Li et al., 2001). We wished to assess 2-LTR circle DNA formation in cells with altered levels of Rad52 expression.

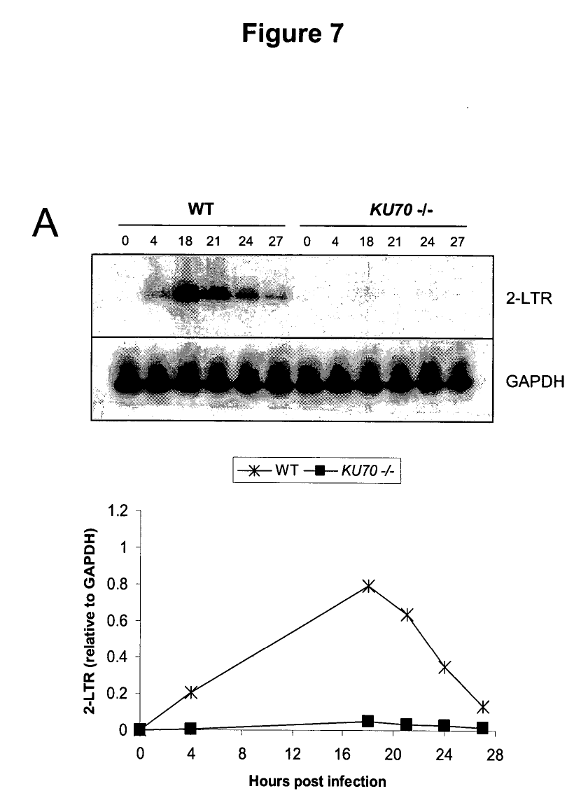

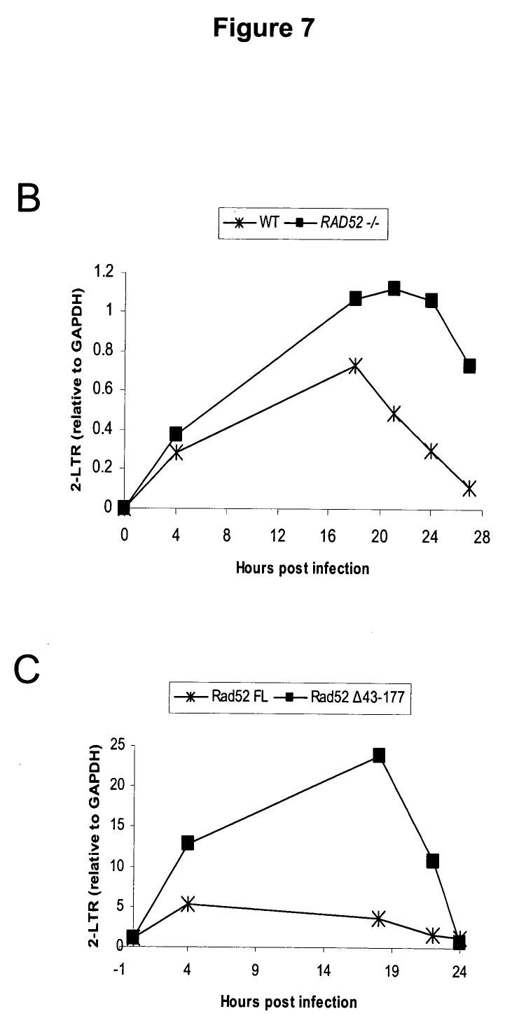

First, we looked at attenuation of 2-LTR circle DNA in NHEJ-defective mouse ES cells. To this end, semi-quantitative PCR analysis of 2-LTR circle DNA from HIV-1 infected KU70^{+/+} or KU70^{-/-} ES cells was performed (FIG. 7A). Total DNA was extracted from ES cells at various time points after HIV-1 infection and the amount of 2-LTR circle DNA estimated by semi-quantitative PCR (see Materials and methods). Analysis of HIV-1 infected ES cells showed that 2-LTR formation was almost completely undetectable in KU70^{-/-} cells in contrast to KU70^{+/+} cells (FIG. 7A).

Similar 2-LTR circle DNA PCR analyses were then performed on HIV-1 infected RAD52^{+/+} or RAD52^{-/-} ES cells (FIG. 7B) and HEK-293 cells transfected with expression plasmids for full-length Rad52 or the Δ43-177 DNA binding deficient mutant of Rad52 (FIG. 7C). Two-LTR circle DNA appeared at a similar rate in both RAD52^{-+/+} and RAD52^{-/-} cells at early times post infection (4 hours) but then accumulated to a higher level in RAD52^{-/-} cells at later time points (particularly noticeable after 18 hours). Two-LTR circle DNA was also maintained at peak levels for longer (18-24 hours) in RAD52^{-/-} cells. In Rad52 overexpressing HEK-293 cells (FIG. 7C) 2-LTR circle DNA formation was significantly impaired by full-length Rad52 but not by the Δ43-177 mutant Rad52. The 43-177 mutant Rad52 has been previously shown not to affect retroviral transduction (FIG. 5). This reduction of 2-LTR circle DNA formation is similar to that observed for KU70^{-/-} ES cells (FIG. 7A).

These data indicate that the loss of RAD52 leads to an up-regulation of HIV-1 2-LTR circle DNA formation, a process thought to be dependent of NHEJ DNA repair, and thus provides indication that the inhibitory activity of RAD52 may proceed through perturbation of the NHEJ repair pathway.

HIV-1 Induced Apoptosis is Unaffected by Rad52 Expression

In addition to HIV-1 2-LTR circle DNA formation, Ku and NHEJ repair has been implicated in protection of cells from retrovirus induced apoptosis. The study by Li et al (2001) suggested 2-LTR circle DNA formation mediated by NHEJ is directly responsible for protection from apoptosis. It was proposed that apoptosis suppression could be achieved through the binding of Ku to the viral cDNA ends and the subsequent activation of the NHEJ DNA repair pathway. The product of this pathway would be the formation of 2-LTR circle DNA, removal of the pro-apoptotic cDNA end and cell survival. If this is indeed the case then we would expect that Rad52, in addition to inhibiting 2-LTR circle DNA formation, would also have the same effect on apoptosis suppression.

We compared the level of HIV-1 vector induced apoptosis in HeLa and Rad52-overexpressing HeLa clones. The proportion of cells undergoing apoptosis was determined by annexin-V staining and live/dead cells discriminated by propidium iodide (PI) counter-staining.

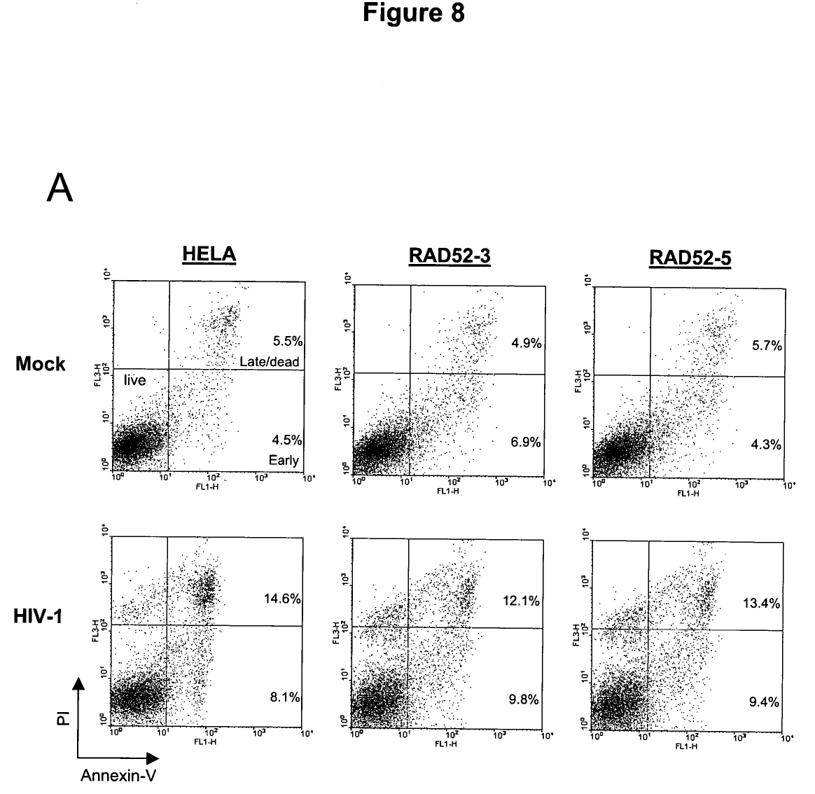

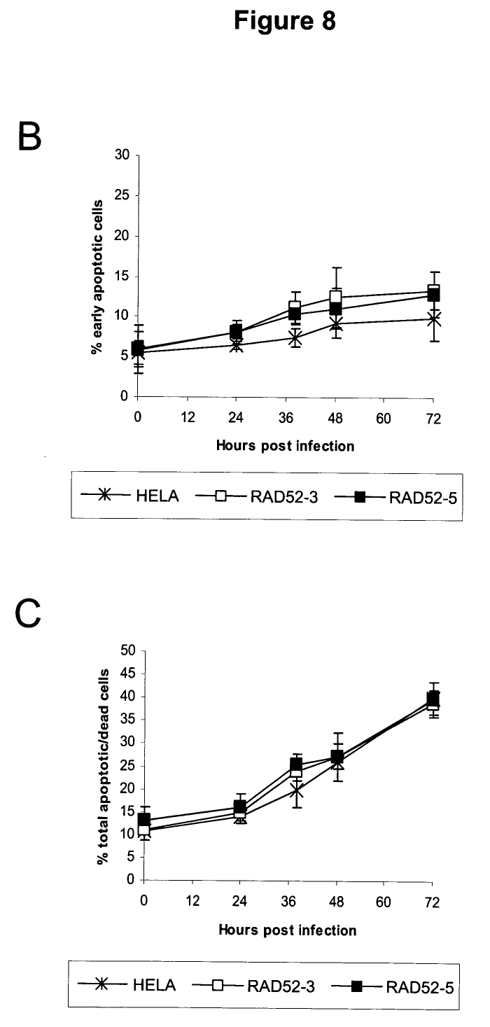

FIG. 8 shows that the extent of apoptosis between HeLa and the Rad52-overexpressing clones was not significantly different. Both early stage apoptosis (high annexin-V but low PI staining) and the total apoptotic/dead (all annexin-V positive cells) cell populations were not increased in either of the two Rad52-overexpressing HeLa clones as might be predicted.

To confirm these results apoptosis/cell death was also assessed using caspase-activation assays and trypan-blue staining. Identical results to annexin-V staining were obtained indicating no differences between the cell lines.

Similar apoptosis analyses using HEK-293 cells transiently transfected with the Rad52 expression plasmid or with RAD52^{-/-} -mouse ES cells also did not show any significant differences in HIV-1 vector induced apoptosis when compared to control cells.

These results indicate that despite its effect on retroviral transduction efficiency and NHEJ mediated 2-LTR circle DNA formation, Rad52 does not influence apoptosis in the cell lines tested. This can be explained by the possibility 2-LTR circle DNA formation and apoptosis suppression are, in fact, not linked. Here, it would be predicted that while Ku and NHEJ repair are required for protection against retroviral induced apoptosis the formation of 2-LTR circle DNA, by itself, is not the mechanism by which it is done. The role of Rad52 in inhibiting retroviral infection is therefore not associated with an increased loss of infected cells through apoptosis, as might be predicted through a simple NHEJ-interference model. The observation that the number of stable HIV-1 integration events can be modulated by Rad52 (FIG. 2) coupled with the knowledge that this is not due to loss through apoptosis (FIG. 7) therefore suggests that integration may be affected directly.

Together, our data provide indication that RAD52 negatively influences the functional interaction of protein factors and complexes with retroviral cDNA ends. The example shown here demonstrates that the recruitment of Ku and the subsequent activation of NHEJ repair, processes known to be required for efficient retroviral infection, can be influenced through competitive binding with Rad52 to retroviral cDNA ends. Although we can show that NHEJ-dependent retroviral events are influenced by Rad52 expression this is unlikely to explain the large effect that Rad52 status has on the efficiency of HIV-1 infection. It is likely that the major function of Rad52 in modulating retroviral infection occurs through interference with other proteins such as integrase or integration co-factors, such as Inil, BAF and hRad18, presumably through competition for retroviral cDNA ends. Studies by Van Dyck et al. (1999) previously suggested a model for control of HR and NHEJ involving potential competition between RAD52 and Ku for DNA ends. Our results are consistent with competition between RAD52 and NHEJ for binding to DNA ends, but the effect on retroviral integration was not suggested by, nor predictable from the proposed model of Van Dyck et al.

Materials and Methods

Materials

The pIRESneo2-HA mammalian expression vector was constructed by insertion of a HA epitope sequence into the EcoRI and NotI sites of pIRESneo2 (Clontech). Full-length human RAD52 cDNA was cloned in-frame with the HA epitope in pIRESneo2HA to yield an N-terminal HA-tagged RAD52 expression plasmid, pIREsneo2-HA-RAD52. The RAD52 deletion mutants Δ43-177 and Δ195-347 were made by restriction enzyme digestion of RAD52 cDNA with Bsu36I-HinDIII and BglII-XbaI respectively, then blunt-ended with Klenow DNA polymerase and re-ligated back together. These deletions maintained the reading frame of RAD52. The deleted RAD52 fragments were then cloned into pIRESneo2-HA as described above.

RAD52 antibody (H-300) was obtained from Santa Cruz Biotechnology, HA antibody (12CA5) was obtained from Boehringer Mannheim and the β-actin antibody (AC-15) was obtained from Sigma. The Ku80 antibody (clone 111) was obtained from NeoMarkers-Labvision Corporation. The FLAG (M2) and β-actin (AC-15) antibodies were obtained from Sigma. Antibodies against Ku70 were generated using routine techniques.

Cell Lines

HELA and HEK-293 cells were grown in Dulbecco's modified Eagle's medium (DMEM) with 10% foetal bovine serum (FBS) (Invitrogen). Mouse embryonic stem (ES) cell line 1B10 (RAD52^{+/+}) and its RAD52^{+/-} and RAD52^{-/-} derivatives (Rijkers et al., 1998), and J1 (KU70^{+/+}) and its KU70^{-/-} derivative (Gu et al., 1997) were grown in the absence of feeder cells on gelatinised dishes in DMEM with 15% FBS and 1000 u/ml leukaemia inhibitory factor (ESGRO, Chemicon International Inc.). V79 (XRCC2⁺) and the XRCC2 defective derivative IRS1 (Jones et al., 1995; Thacker et al., 1995) and AA8 (XRCC3⁺) and its XRCC3 defective derivative IRS1-SF (Tebbs et al., 1995) hamster cell lines were grown in DMEM with 10% FBS. The BRCA2 defective cell line Capan-1 (Abbott et al., 1998) and BxPC3 (BRCA2⁺) control human pancreatic cell lines were grown in RPMI1640 medium with 15% FBS.

HELA-IRES and HELA-RAD52 stable clones were made by transfection of HELA cells with pIRESneo2HA and pIRESneo2-HA-RAD52 plasmids respectively, using Lipofectamine plus reagent (Invitrogen) according to the manufacturers recommended conditions. Individual clones were picked and expanded after growth for 14 days in medium containing 500 μg/ml active G418 (Invitrogen). RAD52 over-expressing clones were identified by immunoblot analysis with both anti-HA and anti-RAD52 antibodies.

Recombinant HIV-1 Vectors and Retrovirus Production

HIV-1 gag/pol expressing packaging constructs LΔP2GPH (Haselhorst et al., 1998), and HIV-1 luciferase transfer vector pHR′Luc and VSV G envelope expression plasmids (Naldini et al., 1996) were used. The integrase D64V mutation (Leavitt et al., 1996) was made by site directed mutagenesis of LΔP2GPH using the Quikchange mutagenesis kit (Stratagene).

Recombinant HIV-1 retroviral stocks were produced using a modification of the transient expression system described by Naldini et al., 1996. Briefly, 6×10⁶ human kidney 293T cells were co-transfected with 10 μg packaging construct LΔP2GPH (IN+ or D64V IN−), 8 μg pHR′-Luc transfer vector and 5 μg VSV G envelope protein expression plasmids using Lipofectamine-2000 reagent (Gibco-BRL). 48 hours post transfection retrovirus-containing cell culture supernatants were harvested, filtered through 0.45 μM cellulose acetate membranes and stored at −80° C. HIV-1 viral titres were estimated using the HIV-1 p24 gag antigen ELISA kit (Beckman-Coulter). Using GFP HIV-1 vectors (pHR′-GFP) it was estimated that 1 ng VSV-G pseudotyped HIV-1 p24 gag corresponds to approximately 601 GFP-transducing units (TU) when titred on HeLa cells.

Retroviral Infection and Luciferase Assays

For HIV-1 luciferase reporter assays (LUCIA) cells were seeded at a density of 2-5×10³ cells per well in 96-well opaque-white tissue culture plates. 24 hours after seeding the media was replaced with retrovirus containing cell culture supernatants at an MOI=0.5 in the presence of 8 μg/ml polybrene. Cells were exposed to virus for 6 hours before being replaced with fresh medium. Luciferase activity was quantified 48 hours post-virus addition on a Packard TopCount-NXT microplate scintillation counter using Bright-Glo luciferase assay reagent (Promega Corporation). For coupled transient RAD52 transfection-retrovirus infection assays HEK-293 cells were seeded at 2×10⁵ cells per well in poly-L-lysine coated 24-well tissue culture plates and allowed to attach for 24 hours. Cells were transfected with a total of 1 μg plasmid DNA using Lipofectamine 2000 reagent (Gibco-BRL) according to the manufacturer's recommended conditions. Transfection efficiencies of >85% were typically observed when using this transfection method. 24 hours post-transfection cells were exposed to retrovirus-containing cell culture supernatants at an MOI=0.5 in the presence of 8 μg/ml polybrene for 6 hours. 48 hours post virus addition cells were lysed in 200 μl 1× passive lysis buffer (PLB; Promega Corporation) and samples split in two for analysis of protein expression by immunoblot and HIV-1 infectivity by luciferase activity. For immunoblot analysis PLB lysates were diluted 1:1 in 2× SDS loading buffer and 20 μl ran in 10% SDS-PAGE gels. Proteins were blotted onto PVDF membranes and probed with anti-HA antibody. Blots were stripped and re-probed with the anti-β-actin antibody as a loading control. Luciferase activity was quantified, in triplicate, with the Dual luciferase reporter (DLR) assay kit (Promega Corporation) using a Turner Designs TD-20/20 luminometer.

Rad52 siRNA Knockdown Transfections

The Rad52 siRNA was designed according to the rules available in the art and judged to be specific though BLAST searching. Rad52 siRNA (target sequence: AAAGACUACCUGAGAUCACUA-SEQ ID NO: 1) and non-specific control siRNA (AAATTCTATCACTAGCGTGAC-SEQ ID NO: 2) were synthesised and pre-duplexed. For HEK-293 plasmid DNA-siRNA co-transfection experiments, cells were transfected in 24-well plates as described above except that 0.5 μg plasmid DNA and 100 nM siRNA duplexes were used. 24, 48 and 72 hours after transfection cells were harvested, washed in PBS and whole cell extracts made by lysing directly in SDS-loading buffer. HA-Rad52 protein expression was determined by immunoblot analysis with anti-HA and β-actin (control) antibodies. For coupled siRNA transfection-retrovirus transduction assays HeLa cells were seeded at 1×10⁵ cells per well in 6-well plates. 100 nM siRNA duplexes were transfected for 4 hours using Oligofectamine reagent (Invitrogen). 24 hours later cells were then transfected again with 100 nM siRNA duplexes and left for a further 24 hours. Cells were trypsin-EDTA harvested, re-seeded into 96-well opaque-white tissue culture plates and incubated for 48 hours. 96-well plate HIV-1 luciferase transduction assays were then performed as described previously.

HIV-1 Chromosomal Immunoprecipitations (ChIPs)

HEK-293 cells were seeded at a density of 6×10⁶ cells per dish in multiple 10-cm poly-L-lysine coated tissue culture dishes and transfected with a total of 24 μg plasmid DNA using Lipofectamine-2000 reagent (Invitrogen). 24 hours post-transfection cells were exposed to retrovirus containing cell culture supernatants at an MOI=0.25 in the presence of 8 μg/ml polybrene for 6 hours. The culture media was then changed and cells incubated for a further 12 hours. DNA-protein interactions were then fixed (cross-linked) by directly adding formaldehyde to a final concentration of 1% and incubating at 37° C. for 10 minutes. Cells were harvested, washed twice in 1×PBS and lysed in sonication buffer (50 mM Tris pH 8.0, 1% SDS, 10 mM EDTA, protease inhibitor cocktail (Boehringer Mannheim)) on ice for 10 minutes. Cell extracts were sonicated four-times in 10 second pulses and cell debris pelleted by centrifugation. Immunoprecipitation of protein-DNA complexes was performed using 2-4 mg of cell supernatants per antibody. Cell supernatants were diluted in IP buffer (20 nM Tris pH8.0, 0.1% SDS, 2 mM EDTA, 1% Triton X-100, 50 mM NaCl, protease inhibitor cocktail) and pre-cleared for 1 hour with Protein-G-Sepharose beads (Amersham-Pharmacia; 50% suspension with 300 μg/ml sssDNA, 0.5 mg/ml BSA in TE). Small aliquots were also taken at this point, protein and DNA extracted and these are referred to as “input” samples. Appropriate antibodies were added at 2-4 μg per mg of pre-cleared cell supernatant and incubated at 4° C. overnight.

Protein-G-Spharose beads were added and incubated for a further 2 hours at 4° C. The sepharose-beads were pelleted by centrifugation, resuspended in IP buffer and were sequentially washed twice in low salt buffer (IP buffer+150 mM NaCl), twice in high salt buffer (IP buffer+500 mM NaCl), once in LiCl wash buffer (10 mM Tris pH 8.0, 1 mM EDTA, 0.5% NP-40, 0.5% sodium deoxycholate, 250 mM LiCl) and twice in TE. Bound protein-DNA complexes were eluted in pre-warmed (65° C.) Elution buffer (50 mM Tris pH8.0, 1% SDS, 100 mM NaHCO₃ 10 mM EDTA) Protein-DNA cross links were reversed by adding NaCl to a final concentration of 200 mM and incubating at 65° C. for 4 hours. DNA was purified by treatment with proteinase-K and phenol:chloroform:IAA extraction then ethanol precipitated in the presence of yeast tRNA carrier. The presence of HIV-1 LTR DNA ends was detected by PCR using primers LTR5 and LTR6 (von Schwedler et al., 1993; Naldini et al., 1996). To allow for semi-quantitative analyses, all PCR reactions were performed using 10-fold serially diluted DNA preparations (1, 10, 100-fold dilutions) and normalised to HIV-1 LTR PCRs of input DNA.

Semi-Quantitative PCR Analysis of HIV-1 cDNA Forms

ES cells were seeded at 2×10⁵ cells per well in gelatinised 6 well plates and allowed to attach for 24 hours. Cells were infected with HIV-1 D64V integrase mutant virus stocks at an MOI=0.5 in the presence of 8 μg/ml polybrene. At 0, 4, 18, 21, 24 and 27 hours after virus addition cells were washed in 1×PBS and harvested by trypsin-EDTA treatment. Cells were washed in 1×PBS then incubated in 300 μl 1×PBS with 100 μg/ml RNase A (Sigma) at room temperature for 10 minutes. Samples were heated at 95° C. for 10 minutes, allowed to cool then digested with 300 μg/ml proteinase K at 56° C. for 2 hours. Insoluble material was pelleted by centrifugation and supernatants transferred to fresh tubes. DNA was precipitated with ethanol and resuspended in 50 μl 10 mM Tris, pH 8.0. PCR analysis of HIV-1 cDNA forms using primers for minus strand strong stop DNA (primers LTR5 and LTR6), late linear DNA (primers LTR5 and NC2) and 2-LTR circle DNA (primers LTR8 and LTR9) were performed as described previously (von Schwedler et al., 1993; Naldini et al., 1996). GAPDH control PCRs were performed under the same conditions as for HIV-1 PCRs. All PCR reactions were limited to 20-26 cycles to ensure linearity of amplification. 1/10th of the PCR reactions were separated on 2% agarose gels, transferred onto nitrocellulose membranes and Southern hybridised with ³²P-labelled (Rediprime kit, Amersham-Pharmacia) HIV-LTR or GAPDH cDNA probes using standard methods. Membranes were exposed to X-ray film at −80° C. and bands quantified by densitometry using the Cyclone phosphoimaging system (Packard).

Semi-Quantitative PCR Analysis of Stably Integrated Proviral DNA

ES cells and the Rad52-overexpressing stable HeLa clones were exposed to retrovirus-containing cell culture supernatants at an MOI=0.5 in the presence of 8 μg/ml polybrene for 6 hours. Transduced cells were propagated, without selection, for 21 days and high MW genomic DNA extracted using Qiagen Blood and Cell Culture (Genomic tip-20) DNA mini kit. Integrated proviral DNA sequences were detected by PCR of the luciferase reporter gene using primers using primers LUC93F (GAGATACGCCCTGGTTCCTG-SEQ ID NO: 3) and LUC-597R (AGAGGAGTTCATGATCAGTGC-SEQ ID NO: 4). GAPDH control and 2-LTR PCRs were performed as previously described. At 21 days after transduction no 2-LTR (unintegrated) DNA could be detected in the DNA preparations. To enable accurate estimates of proviral copy number all PCR reactions were performed on 10-fold serially diluted DNA preparations (1, 10, 100, 1000-fold dilutions) and normalised to GAPDH control PCRs.

Cell Viability and Apoptosis Assays

HeLa and Rad52-overexpressing stable clones were plated on 10-cm dishes at a density of 2×10⁵ cells per dish and transduced with HIV-1 luciferase stocks at an MOI=10. At multiple times after virus addition cells were typsin-EDTA harvested and apoptotic/dead cells stained by incubation with annexin-V and PI (BD Pharmingen) according to the manufacturers recommended conditions. The number of live (annexin-V/PI negative), early apoptotic (annexin-V postitive/PI negative) and late apoptotic/dead (annexin-V/PI positive) cells were quantified by flow cytometric analysis using a BD FACScalibur and CellQuest software.

Discussion

The data presented herein have shown that the homology-directed DSB repair protein RAD52 counteracts retroviral infection in mammalian cells. We obtained a 16-fold increase in retroviral transduction in the absence of RAD52 in mouse ES cells (FIG. 2). Knockdown of Rad52 expression in human cells by RNAi also showed a marked increase in retroviral transduction (FIG. 4) over control cells.

Previously, no overt phenotypes resulting from the lack of RAD52 have been detected. The absence of RAD52 in mice, for example, does not result in DNA damage sensitisation, cancer predisposition, or defects associated with meiotic recombination (Rijkers et al., 1998). In fact, the only defect detected in RAD52^{-/-} ES cells was a modest (30-40%) reduction in homologous gene targeting.

Genetic and biochemical experiments have revealed that RAD52 plays a major role in DSB repair through HR (Paques and Haber, 1999; Kanaar and Hoeijmakers, 1998). Our results indicate that the HR function of RAD52 is not involved in attenuating retroviral infection: other proteins required for HR, such as XRCC2, XRCC3, and BRCA2, do not influence the outcome of HIV-1 infections (either positively or negatively, see FIG. 2). Thus, the HR DSB repair pathway is superfluous with respect to establishing productive retroviral integration.

Two of our observations further support this. Firstly, the domain of RAD52 responsible for interaction with other HR proteins is not required for the suppression of retroviral infection (FIG. 5). Instead, this activity resides in the DNA binding and RAD52-self interaction domain. Secondly, overexpression of RAD52 alone is sufficient for this suppression.

Our studies further demonstrate that the 2-LTR HIV-1 circle DNA formation requires Ku and NHEJ (FIG. 7A; Li et al., 2001; Jeanson et al., 2002), and its formation increases in the absence of RAD52 (FIG. 5B) or decreases when Rad52 is overexpressed (FIG. 5C). This indicates that the expression of RAD52 may interfere with Ku directed NHEJ DNA repair activity.

In addition to 2-LTR circle DNA formation a deficiency in Ku or NHEJ DNA repair leads to increased apoptotic cell death following retroviral infection (Daniel et al., 1999; Li et al., 2001). It has been suggested that the formation of 2-LTR circle DNA and the induction of apoptosis are directly linked. One NHEJ-dependent cell-survival model favoured by Li et al (2001) argues for a direct physical elimination mechanism whereby linear retroviral cDNA ends represent pro-apoptotic signals and that through circularisation by NHEJ these free cDNA ends are removed, protecting the host cell from apoptosis. Based on these findings a direct competition model would be predicted in which in the absence of Ku, or alternatively when Rad52 protein levels are high, activation of the NHEJ pathway does not occur, as determined by the decrease in the number of 2-LTR DNA circles observed, and the host cell undergoes apoptosis, resulting in a non-productive infection.

This simple Rad52-Ku competition model for retroviral suppression does not fit with our experimental observations.

Our own studies on NHEJ repair show that KU70^{-/-} cells have impaired 2-LTR circle DNA formation (FIG. 7A) and enhanced susceptibility towards HIV-1 induced apoptosis. However, when looking in either RAD52^{-/-} or in Rad52-overexpressing HeLa cells there were no substantial differences observed in the level of HIV-1 induced cell killing when compared to control cells (FIG. 8). Paradoxically, this means that Rad52 affects the end-joining activity of Ku yet does not interfere with Ku-dependent protection from retrovirus cDNA-induced apoptosis. This disparity could only be explained if either removal of the pro-apoptotic linear retroviral cDNA through LTR circle formation is not required for apoptosis suppression or that linear retroviral DNA can be removed by alternative compensatory pathways.

It has been shown that Ku, DNA-PKcs, XRCC4 and DNA ligase-IV, all components of the NHEJ DNA repair complex, are associated with retrovirus-induced apoptosis suppression (Daniel et al., 1999) and 2-LTR formation (Li et al., 2001; Jeanson et al., 2002). Therefore, the formation of the complete or active NHEJ repair complex at the cDNA end alone, but not actual DNA end-joining itself, is sufficient to impair signalling to undergo apoptosis.

Rad52 when bound to retroviral cDNA ends would prevent NHEJ complex formation, presumably through interference with Ku, loss of end-joining activity and impaired 2-LTR circle formation. Retroviral cDNA ends when bound to Rad52 interactions would also be prevented from being detected as a DNA damage site and would not invoke an apoptotic response. Regardless of whether Ku or Rad52 was bound, apoptosis would still be suppressed and no changes in apoptosis susceptibility observed.

Our results show that the inhibitory activity of Rad52 on retroviral infection cannot be explained through modulation of cell survival through inhibition of NHEJ DNA repair, as might be predicted from studies by Van Dyck et al (1999) or Li et al (2001). It would be envisioned that Rad52 prevents the association of other retroviral cDNA end-binding proteins, in addition to Ku, that form part of the pre-integration complex (Bowerman et al., 1989). Candidate proteins could include integration co-factors such as Inil (hSNF5; Kalpana et al., 1994), HMG I(Y) (HMGA1; Farnet and Bushman, 1997), BAF (Lee and Craigie, 1998), and integrase itself. Recently, the post-replication/translesion DNA repair protein hRad18 has also been shown to interact with HIV-1 integrase (Mulder et al, 2002). Studies in yeast have shown that DNA pathway switching can be influenced by Srs2 helicase though modulating RAD18 and RAD52 interactions (Broomfield et al., 2001). This observation may provide an interesting link between Rad52 and HIV-1 integration.

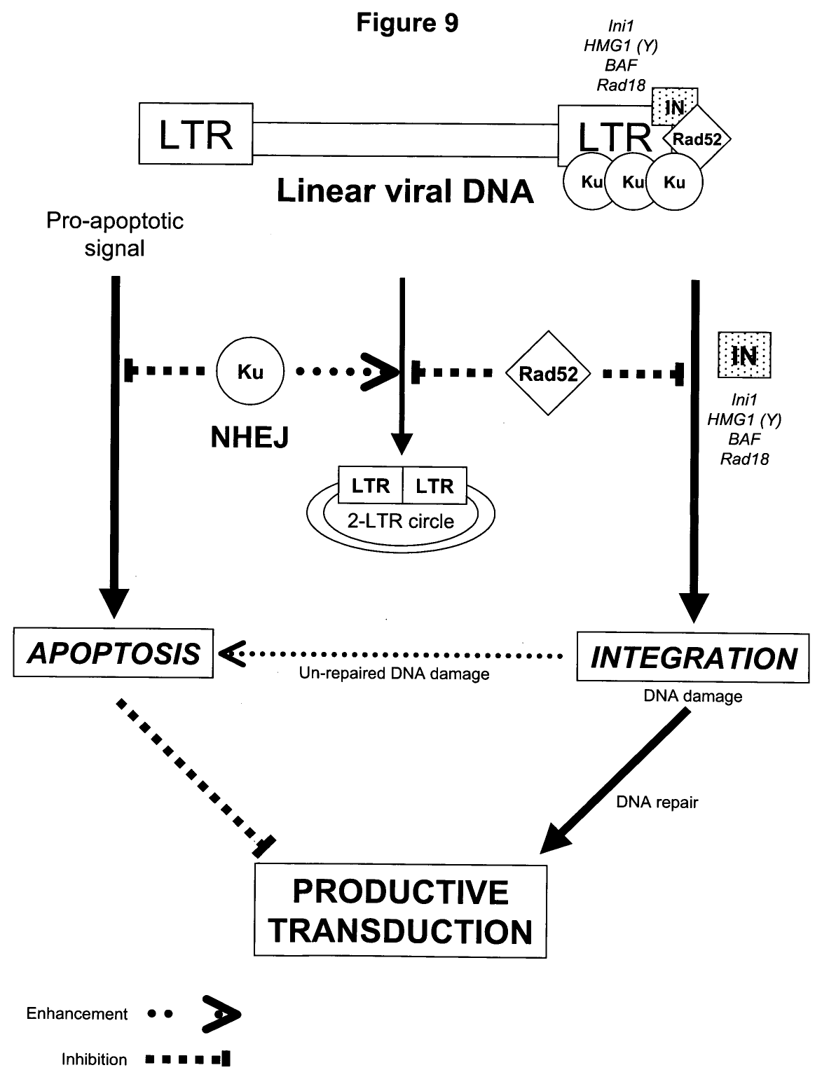

Based on our data, a model for the effect of Rad52 on retroviral infection can be invoked which is depicted in FIG. 9. Here, suppression of retroviral infection by Rad52 may be due to capping of retroviral cDNA ends and blocking the loading or recruitment of other proteins or complexes required for efficient integration. The major influence of Rad52 is through inhibition of integration and not though enhancement of apoptosis with 2-LTR circle DNA formation a side-product of Ku and retrovirus cDNA end-binding interactions. Although Ku is a DNA end-binding protein it is known that multiple Ku subunits can load onto and translocate into the DNA strand (Yoo and Dynan 1999). This is significant as it may be why Ku does not exhibit the same inhibitory effects as Rad52 despite being an active part of the pre-integration complex. Ku, when bound internally onto the retroviral cDNA may not interfere with the association of other DNA-end binding proteins such as integrase. Indeed, studies have demonstrated that Ku does not affect the cleavage or strand-transfer activities of integrase in-vitro (Li et al., 2001).

Studies in S. cerevisiae have shown that HR DNA repair and the RAD52 epistasis group of proteins are involved in regulating Tyl element retrotransposition (Rattray et al., 2000). Yeast Tyl retrotransposons are LTR-containing retroelements that are functionally and structurally related to retroviruses and are the most abundant repetitive sequences found in the S. cerevisiae genome (see Boeke and Sandmeyer, 1991 for review). By using HR defective yeast strains, Rattray et al. (2000) showed that loss of yeast Rad51 or RAD52 led to an increase in Tyl retrotransposition by 11- and 24-fold respectively, when compared to wild type strains. The increase in retrotransposition was also linked to an increase in unincorporated Ty1 element cDNA. The authors suggest that the role of HR DNA repair is to effectively suppress Ty1 element retrotransposition within the yeast genome. S. cerevisiae Ty1 retrotransposition has also been shown to rely on the NHEJ repair pathway for efficient integration (Downs and Jackson, 1999). The involvement of both suppressive HR and productive NHEJ DNA repair pathways in yeast Ty1 retrotransposition is indicative of competition between these events. Although parallels between the DNA repair requirements of yeast retrotransposons and mammalian retroviruses have emerged, significant differences between the two can also be seen. The suppression of yeast Ty1 retrotransposition by RAD52 is consistent with our data for retroviruses. However, the lack of a function for other HR repair proteins, such as Rad51, in influencing retroviral infection may highlight the differences in DNA DSB repair pathway utilisation and redundancy in mammalian systems.

Very recently, a study has demonstrated competition between RAD52 and Ku in modulating transduction of cells with recombinant adeno-associated virus (rAAV) (Zentilin et al., 2001). This work showed that rAAV transduction is promoted in Ku-defective cells but inhibited in RAD52 knockout cells, the exact opposite of the results now observed for retroviral transductions (rAAV is not a retrovirus). However, in rAAV transduction, large circular virus concatamers are thought to be the pre-integration intermediates (Duan et al., 1998, Yang et al., 1999). In the light of the experimental work presented for the first time herein, the competitive involvement of Ku and RAD52 proteins in both rAAV and retrovirus transduction suggests that the NHEJ and HR DNA repair pathways may represent common cellular targets, hijacked by viruses to complete their infectious cycles. Modulation of NHEJ or HR DNA repair proteins therefore provides the potential to regulate viral infection. For example, by targeting the NHEJ pathway and its associated signalling pathways retroviral infection may be repressed. Alternatively, inhibition of RAD52 gene expression may be used to achieve a considerable enhancement of viral vector-based gene transduction, significantly increasing the potential of retroviral-based gene therapy.

### REFERENCES

Abbott et al. (1998) J Natl Cancer Inst, 90, 978-85.

Boeke and Sandmeyer (1991) Yeast transposable elements. Cold Spring Harbor Laboratory Press, Cold Spring Harbor, N.Y.

Broomfield et al. (2001) Mutat Res, 486, 167-84.

Brown (1990) Curr Top Microbiol Immunol, 157, 19-48.

Coffin et al. (1997) Retroviruses. Cold Spring Harbor Laboratory Press, Cold Spring Harbor, N.Y.

Cox et al. (2000) Nature. 404, 37-41.

Daniel et al. (1999) Science, 284, 644-7.

Daniel et al. (2001) Mol Cell Biol, 21, 1164-72.

Daniel et al. (2002). Proc Natl Acad Sci USA, 100, 4778-83.

Davies et al. (2001) Mol Cell. 7, 273-82.

Downs and Jackson (1999) Mol Cell Biol, 19, 6260-8.

Duan et al. (1998) J Virol, 72, 8568-77.

Farnet and Bushman (1997) Cell, 88, 483-92.

Gaken et al. (1996) J Virol, 70, 3992-4000.

Game. (1993) Semin Cancer Biol, 4, 73-83.

Gu et al. (1997) Proc Natl Acad Sci USA, 94, 8076-81.

Ha et al. (2001) Proc Natl Acad Sci USA, 98, 3364-8.

Haselhorst et al. (1998) J Gen Virol, 79, 231-7.

Hawley. (2001) Curr Gene Ther, 1, 1-17.

Huang et al. (1996) Proc Natl Acad Sci USA, 93, 4827-32.

Jeanson et al. (2002) Virology, 300, 100-8.

Johnson and Jasin (2000) Embo J, 19, 3398-407.

Jones et al. (1995) Genomics, 26, 619-22.

Kalpana et al. (1994) Science, 266, 2002-6.

Kanaar and Hoeijmakers (1998) Nature. 391, 335, 337-8

Khanna and Jackson (2001) Nat Genet, 27, 247-54.

Lee and Craigie (1998) Proc Natl Acad Sci USA, 95, 1528-33.

Leavitt et al. (1996) J Virol, 70, 721-8.

Li et al. (2001) Embo J, 20, 3272-81.

Liang et al. (1998) Proc Natl Acad Sci USA, 95, 5172-7.

Lim and Hasty. (1996) Mol Cell Biol, 16, 7133-43.

Lin, C. W. and Engelman, A. (2003) J Virol, 77, 5030-6

Milne and Weaver (1993) Genes Dev, 7, 1755-65.

Moynahan et al. (1999) Mol Cell. 4, 511-8.

Moynahan et al. (2001) Mol Cell. 7, 263-72.

Mulder et al. (2002) J Biol Chem, 277, 27489-93.

Naldini et al. (1996) Science, 272, 263-7.

O'Regan et al. (2001) J Biol Chem, 276, 22148-53.

Paques and Haber (1999) Microbiol Mol Biol Rev, 63, 349-404.

Park et al. (1996) J Biol Chem, 271, 18996-9000.

Petes et al. (1991) In The Molecular and Cellular Biology of the Yeast Saccharomyces. Cold Spring Harbor Press, Cold Spring Harbor, N.Y., pp. 407-521.

Pierce et al (2001) Genes Dev, 15, 3237-42.

Rattray et al. (2000) Genetics, 154, 543-56.

Rich et al. (2000) Nature, 407, 777-83.

Rijkers et al. (1998) Mol Cell Biol, 18, 6423-9.

Rothstein et al. (2000) Genes Dev. 14,1-10.

Shen et al. (1996a) Mutat Res, 364, 81-9.

Shen et al. (1996b) J Biol Chem, 271, 148-52.

Scherr and Eder. (2002) Curr Gene Ther, 2, 45-55.

Smith and Jackson (1999) Genes & Dev, 13, 916-934.

Sonoda et al. (1998) Embo J, 17, 598-608.

Takata et al. (2001) Mol Cell Biol, 21, 2858-66.

Tebbs et al. (1995) Proc Natl Acad Sci USA, 92, 6354-8.

Thacker et al. (1995) Hum Mol Genet, 4, 113-20.

Thompson et al. (2001) Mutat Res. 477, 131-53.

Tsuzuki et al. (1996) Proc Natl Acad Sci USA, 93, 6236-40.

Van Dyck et al. (1999) Nature, 398, 728-31.

Van Gent et al. (2001) Nat Rev Genet, 2, 196-206.

von Schwedler et al. (1993) J Virol, 67, 4945-55.

Yang et al. (1999) J Virol, 73, 9468-77.

Zentilin et al. (2001) J Virol, 75, 12279-87.

All combinations of features of the attached claims are to be considered disclosed herein.

1. A method of promoting integration of a retroviral vector into the genome of a mammalian cell into which the retroviral vector is introduced, the method comprising inhibiting RAD52 DNA-binding activity in the cell.

2. A method according to claim 1 comprising inhibiting RAD52 DNA-binding activity in the cell by inhibiting production of RAD52 protein.

3. A method according to claim 1 comprising inhibiting RAD52 DNA-binding activity in the cell by inhibiting binding of DNA by RAD52.

4. A method according to claim 2 comprising providing to the cell double-stranded RNAi.

5. A method according to claim 2 comprising providing to the cell antisense RNA.

6. A method according to claim 3 comprising providing to the cell a molecule that binds RAD52 protein.

7. A method according to claim 1 comprising temporarily inhibiting RAD52 DNA-binding activity in the cell.

8. A method according to claim 1 wherein the mammalian cell is a cell-line in culture.

9. A method according to claim 1 wherein the mammalian cell is ex vivo.

10. A method according to claim 9 comprising introducing a retroviral vector into a cell removed from a mammal and inhibiting RAD52 DNA-binding activity in the cell.

11. A method according to claim 9 wherein the cell is a stem cell.

12. A method of obtaining an agent that promotes retroviral integration into the genome of a mammalian cell, the method comprising:
selecting one or more test substances that bind RAD52 protein and/or inhibit RAD52 binding to DNA;
testing the test substance or substances for ability to promote retroviral integration into the genome of a mammalian cell, by providing each test substance within a mammalian cell into which a retroviral vector is introduced and determining a change in retroviral integration into the genome of the mammalian cell compared with a control experiment,
wherein an increase in retroviral integration compared with the control experiment is indicative of ability of the test substance to promote retroviral integration into the genome of a mammalian cell and said agent is thereby obtained.

13. A method according to claim 12 comprising obtaining one or more test substances that bind RAD52 protein by contacting RAD52 protein or a DNA binding fragment thereof with test substances and selecting one or more of the test substances that bind RAD52 protein or the DNA binding fragment thereof.

14. A method according to claim 12 further comprising formulating the obtained agent into a composition comprising at least one additional component.

15. A method of obtaining an agent that promotes retroviral integration into the genome of a mammalian cell, the method comprising:
selecting one or more test substances that comprise RNA with nucleotide sequence complementary to a mammalian RAD52 gene sequence, which RNA is dsRNA or antisense RNA or is a ribozyme specific for a mammalian RAD52 gene sequence;
testing the test substance or substances for ability to promote retroviral integration into the genome of a mammalian cell, by providing each test substance within a mammalian cell into which a retroviral vector is introduced and determining a change in retroviral integration into the genome of the mammalian cell compared with a control experiment,
wherein an increase in retroviral integration compared with the control experiment is indicative of ability of the test substance to promote retroviral integration into the genome of a mammalian cell and said agent is thereby obtained.

16. A method according to claim 15 further comprising formulating the obtained agent into a composition comprising at least one additional component.

17. A method of inhibiting retroviral integration in a mammalian cell, the method comprising increasing mammalian RAD52 DNA-binding activity in the cell.

18. A method according to claim 17 comprising causing overexpression of mammalian RAD52 protein or a DNA-binding fragment thereof within the cell.

19. A method according to claim 18 comprising introducing into the cell nucleic acid encoding mammalian RAD52 protein or a DNA-binding fragment thereof.

21. A method according to claim 17 wherein the cell is in vitro or ex vivo.


SEQUENCE LISTING (4 sequences)

SEQ ID NO: 1 - RNA, 21 nt, Artificial Sequence
  synthetic siRNA sequence
aaagacuaccugagaucacua

SEQ ID NO: 2 - DNA, 21 bp, Artificial Sequence
  synthetic siRNA control sequence
aaattctatcactagcgtgac

SEQ ID NO: 3 - DNA, 20 bp, Artificial Sequence
  reporter gene primer
gagatacgccctggttcctg

SEQ ID NO: 4 - DNA, 21 bp, Artificial Sequence
  reporter gene primer
agaggagttcatgatcagtgc